(19) United States
(12) Patent Application Publication
(10) Pub. No.: US 2026/0084222 A1
(43) Pub. Date: Mar. 26, 2026

(54) MECHANICAL SURGICAL ARM

(71) Applicant: Alphatec Spine, Inc., Carlsbad, CA (US)

(72) Inventors: Kyle Stewart, Oceanside, CA (US); Jon Costabile, Del Mar, CA (US); Andrew Morris, San Diego, CA (US)

(21) Appl. No.: 19/337,796

(22) Filed: Sep. 23, 2025

Related U.S. Application Data
(60) Provisional application No. 63/699,148, filed on Sep. 25, 2024.

(51) Int. Cl. B23B 31/20; A61B 46/10

(52) U.S. Cl. CPC B23B 31/20; A61B 46/10

(57) ABSTRACT

This disclosure relates generally to mechanical surgical arms having a connector for attaching a surgical tool to the arm across a sterile drape. In various aspects, a drape connector for securing a tool to a surgical arm across a sterile drape includes a flexible collet for receiving a ball of a connection shank of the surgical arm to selectively secure the flexible collet to the surgical arm. The drape connector may also include a lock associated with an inferior end of the flexible collet. The lock may be for adjusting the flexible collet from an unlocked position to a locked position. Further, the drape connector may include a housing for receiving the flexible collet and the lock. The housing defines a window to provide access to the lock to adjust the flexible collet from the unlocked to the locked position.

### CROSS REFERENCE TO RELATED MATTER

The present application claims priority from U.S. Provisional Application No. 63/699,148, filed Sep. 25, 2024, the entirety of which is incorporated herein by reference.

### TECHNICAL FIELD

This disclosure relates generally to mechanical surgical arms, and in some aspects to mechanical surgical arms having a connector for attaching a surgical tool to the arm across a sterile drape.

### SUMMARY

Disclosed are systems, devices, and/or methods of use thereof regarding mechanical surgical arms having a connector for attaching a surgical tool to the arm across a sterile drape. In various aspects, a drape connector for securing a tool to a surgical arm across a sterile drape includes a flexible collet for receiving a ball of a tool connection shank of the surgical arm to selectively secure the drape connector to the surgical arm. The drape connector may also include a lock associated with an inferior end of the flexible collet. The lock may be for adjusting the flexible collet from an unlocked position to a locked position. Further, the drape connector may include a housing for receiving the flexible collet and the lock. The housing may define a window to provide external access to the lock to adjust the flexible collet from the unlocked position to the locked position.

In various aspects, a system for connecting a drape to a surgical arm includes a surgical arm having a tool connection shank with a ball at an end of the surgical arm. The system may also include a drape for placing between the tool connection shank and a drape connector. The drape connector may be for securing a tool the surgical arm. The drape connector may include a flexible collet for selectively receiving the ball of the tool connection shank to secure the drape connector to the surgical arm across the drape, a lock for adjusting the flexible collect from an unlocked position to a locked position, and housing for receiving the flexible collet and the lock. The housing may define a window to provide external access to the lock in order to adjust the flexible collet from the unlocked position to the locked position.

In various aspects, a method of connecting a surgical tool to a surgical arm may include draping the surgical arm with a sterile drape and attaching a first end of a drape connector to the surgical arm and over the sterile drape. The drape connector may have a flexible collet in an open position. The method may also include actuating a lock of the drape connector, such that the flexible collet is in a closed position and locked to the surgical arm. Additionally, the method may include attaching the surgical tool to a second end of the drape connector opposite the first end.

In various aspects, a mechanical arm for use in surgery includes a distal joint having a handle and a drape connector. The drape connector may be for connecting a surgical instrument to the mechanical arm across a sterile drape. The distal joint may have three (3) degrees of freedom allowing the surgical instrument to be rotated, translated forward and backward, and angularly adjusted. The mechanical arm may also include a medial joint connected to the distal joint, with the medial joint having one (1) degree of freedom allowing the distal joint to pivot about the medial joint. Further, the mechanical arm may include a translating link connected to the medial joint and extending inferiorly away from the distal joint. The translating link may have one (1) degree of freedom allowing a length of the mechanical arm to be adjusted. Still further, the mechanical arm may include a proximal joint connected to the translating link, with the proximal joint having two (2) degrees of freedom, the proximal joint for connecting the mechanical arm to a surgical table. Each of the distal joint, the medial joint, and the proximal joint may be transitional between a default locked state and an open state.

In various aspects, a method of using a mechanical surgical arm includes opening a high pressure fluid line to actuate an actuator within a distal joint, causing a housing of the distal joint to mechanically separate from a clamp of the distal joint and allowing three (3) degrees of freedom for the distal joint. The method may also include compressing a spring of a medial joint, where the medial joint is connected to the distal joint. Compression of the spring may cause a fixed taper flange of the medial joint to mechanically separate from a floating taper flange of the medial joint and allow the distal joint to pivot about the medial joint. Additionally, the method may include rotating a surgical instrument connected to the distal joint.

In various aspects, a method of attaching a surgical instrument to a mechanical arm includes aligning a receiver of the mechanical arm over a basket, the basket held by a housing and joining the receiver to the basket. The receiver may be contained within a sterile drape and the basket may be external to the sterile drape, such that the receiver is joined to the basket across the sterile drape. The method may also include locking the basket to the receiver and attaching a surgical arm to the housing.

In various aspects, a mechanical arm for use in surgery includes a distal joint having a handle and a drape connector. The drape connector may be for connecting a surgical instrument to the mechanical arm across a sterile drape. The distal joint may have three (3) degrees of freedom allowing the surgical instrument to be rotated, translated forward and backward, and angularly adjusted. The mechanical arm may also include a medial joint connected to the distal joint, with the medial joint having one (1) degree of freedom allowing the distal joint to pivot about the medial joint. Further, the mechanical arm may include a translating link connected to the medial joint and extending inferiorly away from the distal joint. The translating link may have one (1) degree of freedom allowing a length of the mechanical arm to be adjusted. Still further, the mechanical arm may include a proximal joint connected to the translating link, with the proximal joint having two (2) degrees of freedom, the proximal joint for connecting the mechanical arm to a surgical table. Each of the distal joint, the medial joint, and the proximal joint may include a fixed and a floating taper locked together by spring forces. Additionally, each of the distal joint, the medial joint, and the proximal joint may be transitional between a default locked state and an open state only by pneumatic actuators using high pressure CO₂ to remove compression on the floating taper and move each of the distal joint, the medial joint, and the proximal joint to the open state.

In various aspects, a drape connector for sterilely connecting a tool to a surgical arm having a tool connection shank includes a flexible collet for receiving a ball of a tool connection shank of the surgical arm to selectively secure the drape connector to the surgical arm across a sterile drape. The drape connector may also include a lock associated with an inferior end of the flexible collet. The lock may be for adjusting the flexible collet from an unlocked position to a locked position. Further, the drape connector may include a housing for receiving the flexible collet and the lock. A superior end of the housing may have a plurality of teeth and a plurality of cut-outs. The superior end of the housing may be for mechanically engaging the tool connection shank, such that the superior end of the housing and the tool connection shank have six points of contact between the superior end of the housing and the tool connection shank. The housing may define a window to provide external access to the lock to adjust the flexible collet from the unlocked position to the locked position.

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 illustrates a perspective view of a mechanical arm for use in surgery;

FIG. 2 illustrates a back view of the mechanical arm of FIG. 1;

FIG. 3 illustrates a perspective view of the distal joint of the mechanical arm of FIGS. 1 and 2;

FIG. 4 illustrates a side view of the distal joint of FIG. 3;

FIG. 5 illustrates a perspective view of the distal joint of FIG. 3, with the handle, link, and drape connector removed;

FIG. 6 illustrates an exploded view of FIG. 5;

FIG. 7 illustrates the pneumatic assembly from the distal joint of FIG. 5, with the housing and outer clamp removed;

FIG. 8 illustrates a cross-sectional view of the distal joint of FIGS. 1 through 5, with the handle and drape connector removed;

FIG. 9 illustrates a perspective, cross-sectional view of FIG. 7;

FIG. 10 illustrates a perspective cross-sectional view of the housing, outer clamp, and inner clamp from the distal joint of FIGS. 3 through 8;

FIG. 11 illustrates a perspective view of the drape connector from the mechanical arm of FIGS. 1 and 2;

FIG. 12 illustrates a partially exploded view of the drape connector of FIG. 11;

FIG. 13 illustrates another partially exploded view of the drape connector of FIG. 11;

FIG. 14 illustrates an exploded view of the drape connector of FIG. 11, with the tool connection shank removed;

FIG. 15 illustrates a cross-sectional view of the drape connector of FIG. 11;

FIG. 16 sequentially illustrates connection of the tool connection shank to the drape connector;

FIG. 17 illustrates a perspective view of the medial joint of the mechanical arm of FIGS. 1 and 2;

FIG. 18 illustrates an exploded view of FIG. 17;

FIG. 19 illustrates the floating taper flange of the medial joint of FIG. 17 and the distal joint stem connecting the medial joint to the distal joint;

FIG. 20 illustrates a cross-sectional view of the medial joint of FIG. 17;

FIG. 21 illustrates another cross-sectional view of the medial joint of FIG. 17;

FIG. 22 illustrates a perspective view of the translating link of the mechanical arm of FIGS. 1 and 2;

FIG. 23 illustrates a cross-sectional view of the translating link of FIG. 22;

FIG. 24 illustrates another cross-sectional view of the translating link of FIG. 22;

FIG. 25 illustrates a perspective view of the proximal joint from the mechanical arm of FIGS. 1 and 2;

FIG. 26 illustrates another perspective view of the proximal joint;

FIG. 27 illustrates an exploded view of a medial-lateral portion of the proximal joint;

FIG. 28 illustrates a perspective, cross-sectional view of the medial-lateral portion of the proximal joint;

FIG. 29 illustrates an exploded view of a cranial-caudal portion of the proximal joint;

FIG. 30 illustrates a perspective, cross-sectional view of the cranial-caudal portion of the proximal joint;

FIG. 31 illustrates a cross-sectional view of the proximal joint connected to the translating link;

FIG. 32 illustrates a perspective view of the handle from the mechanical arm of FIGS. 1 and 2;

FIG. 33 illustrates a cross-sectional view of the handle;

FIG. 34 illustrates another cross-sectional view of the handle;

FIG. 35 illustrates a perspective view of the inferior end of the handle;

FIGS. 36 through 38 are flowcharts of example methods of the present disclosure

FIG. 39 illustrates another embodiment of a handle and drape connector for connecting a surgical instrument to the mechanical arm of FIGS. 1 and 2;

FIG. 40 illustrates the handle and drape connector of FIG. 39 having a surgical instrument attached thereto;

FIG. 41 illustrates the drape connector of FIG. 39;

FIG. 42 illustrates a cross-sectional view of the drape connector of FIG. 39 taken through the line A-A;

FIG. 43 illustrates a cross-sectional view of the drape connector of FIG. 39 taken through the line B-B;

FIGS. 44A and 44B illustrate another embodiment of a connection shank for connecting the drape connector of FIGS. 39 through 43 with the distal joint of the mechanical arm;

FIG. 45 illustrates the handle and drape connector of FIGS. 39 through 43 connected to the distal joint across a sterile drape through the connection shank of FIGS. 44A and 44B;

FIGS. 46 and 47 illustrate another embodiment of a handle and drape connector for connecting a surgical instrument to the mechanical arm of FIGS. 1 and 2, with an attached surgical instrument;

FIG. 48 illustrates the handle and drape connector of FIGS. 46 and 47;

FIG. 49 illustrates the handle and drape connector of FIGS. 46 through 48 connected to the distal joint across a sterile drape through the connection shank of FIGS. 44A and 44B;

FIGS. 50 and 51 illustrate another embodiment of a drape connector for connecting a surgical instrument to the mechanical arm of FIGS. 1 and 2, with an attached surgical instrument;

FIG. 52 illustrates the surgical instrument of FIGS. 50 and 51;

FIG. 53 illustrates a second connection shank for connecting the surgical instrument of FIGS. 50 through 52 with the distal joint of the mechanical arm of FIGS. 1 and 2;

FIG. 54A illustrates another embodiment of a handle and drape connector for connecting a surgical instrument to the mechanical arm of FIGS. 1 and 2;

FIG. 54B illustrates a proximal end of the drape connector of FIG. 54A, with a flexible collet received and locked within the drape connector;

FIG. 54C illustrates a cross-sectional view of the drape connector with the flexible collet in the locked position;

FIG. 55A illustrates the proximal end of the drape connector of FIG. 54A, with the flexible collet received by the drape connector in an unlocked position; and

FIG. 55B illustrates a cross-sectional view of the drape connector with the flexible collet in the open position.

### DETAILED DESCRIPTION

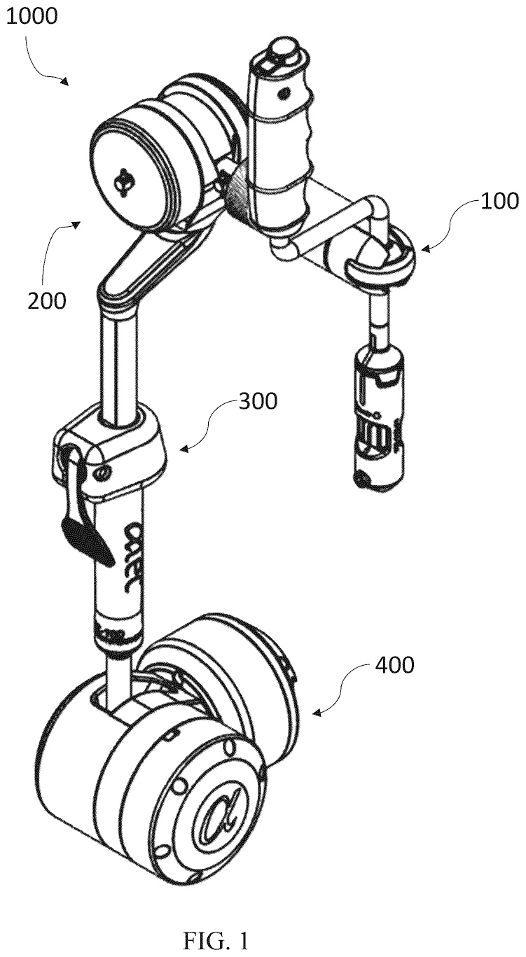

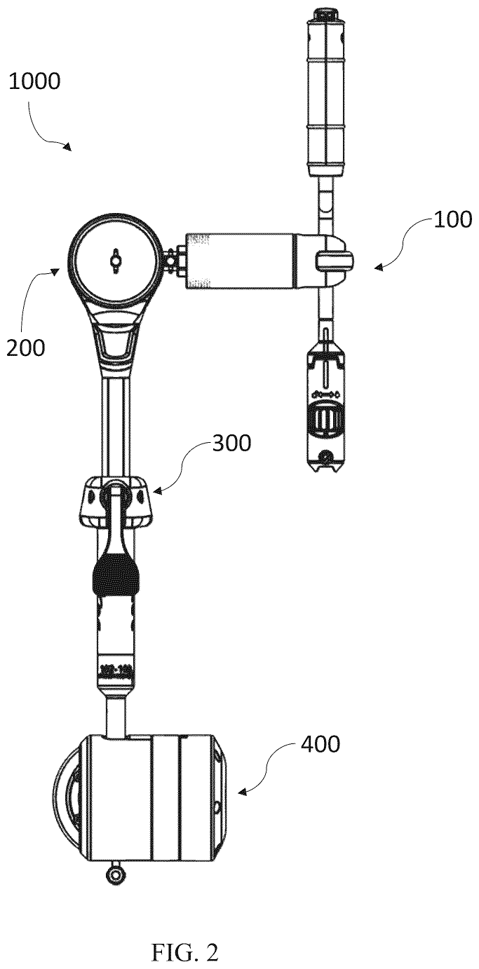

FIGS. 1 and 2 illustrate a mechanical arm 1000 for use in surgical procedures, such as spinal or other surgeries. As illustrated, the mechanical arm 1000 includes a distal joint 100, a medial joint 200 connected to the distal joint 100, a translating link 300 connected to the medial joint 200, and a proximal joint 400 connected to the translating link 300. The mechanical arm 1000 may be covered by a sterile drape or covering (not illustrated) during a surgical procedure.

Each of the distal joint 100, the medial joint 200, the translating link 300, and the proximal joint 400 are in a closed or locked state or closed or locked configuration by default. The mechanical arm 1000 may be connectable to a high-pressure fluid line (e.g., high-pressure CO₂ or another high-pressure fluid) to pneumatically open one or more of the distal joint 100, the medial joint 200, the translating link 300, and/or the proximal joint 400. Alternatively, one or more of the distal joint 100, the medial joint 200, the translating link 300, and/or the proximal joint 40 may be mechanically moveable without high-pressure fluid. In one embodiment, the distal joint 100, the medial joint 200, and/or the proximal joint 400 are only opened through pneumatic actuation, and the translating link 300 is a mechanical joint that is open without pneumatic actuation.

The distal joint 100 is for attaching a surgical tool to the mechanical arm 1000 and maneuvering the surgical tool during a surgical procedure. When open, the distal joint 100 may provide three (3) simultaneous degrees of freedom or motion for the surgical tool: rotation of the tool; angular adjustment or movement of the surgical tool; and, forward and backwards translation of the surgical tool. The surgical tool may be attached to the distal joint 100 across a sterile drape, without materially tearing or puncturing the drape. In this way, the sterility of the mechanical arm 1000 can be maintained across multiple surgical procedures. Accordingly, the mechanical arm 1000 can be used for multiple surgical procedures without requiring sterilization.

When open, the medial joint 200 may provide one (1) degree of freedom allowing the distal joint 100 to pivot about the medial joint 200. When open, the translating link 300 provides one (1) degree of freedom allowing a length of the mechanical arm 1000 to be adjusted. The proximal joint 400 is for connecting the mechanical arm 1000 to a surgical table. When open, the proximal joint 400 provides two (2) degrees of freedom, allowing the mechanical arm 1000 (e.g., translating link 300) to pivot or rotate about the proximal joint 400 and to be translated forward and/or backward.

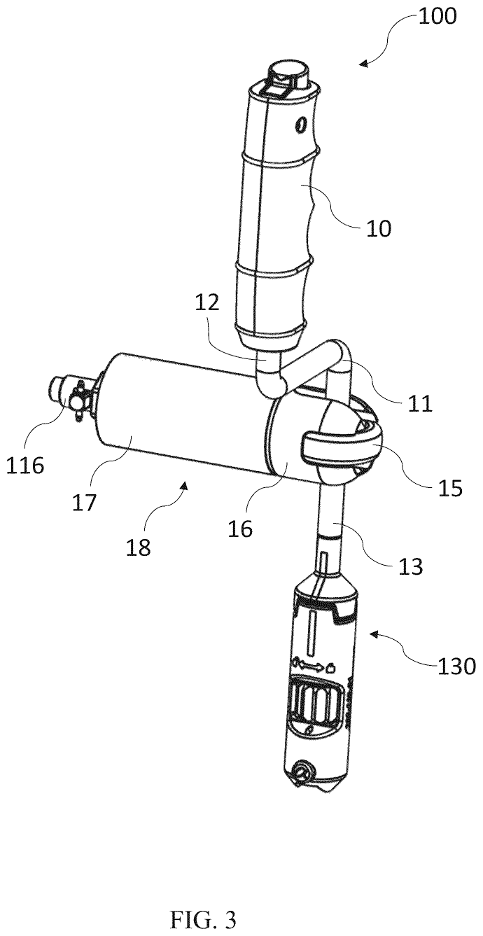

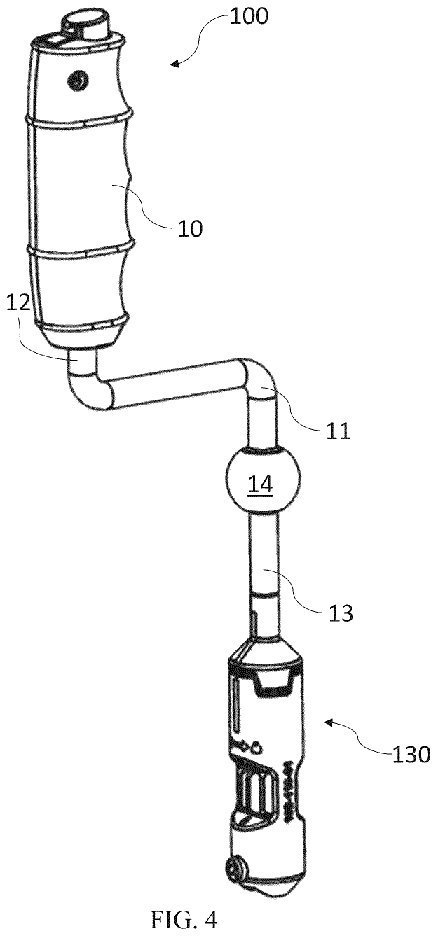

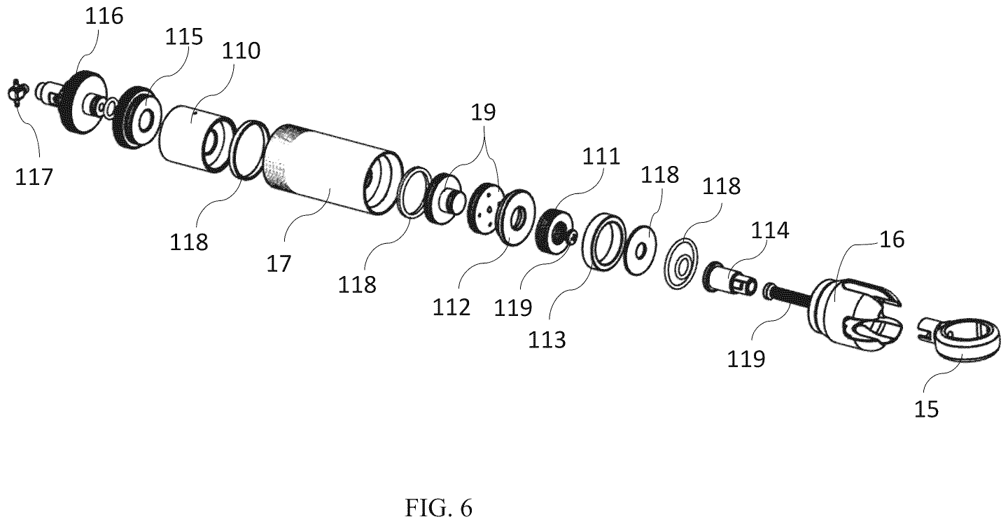

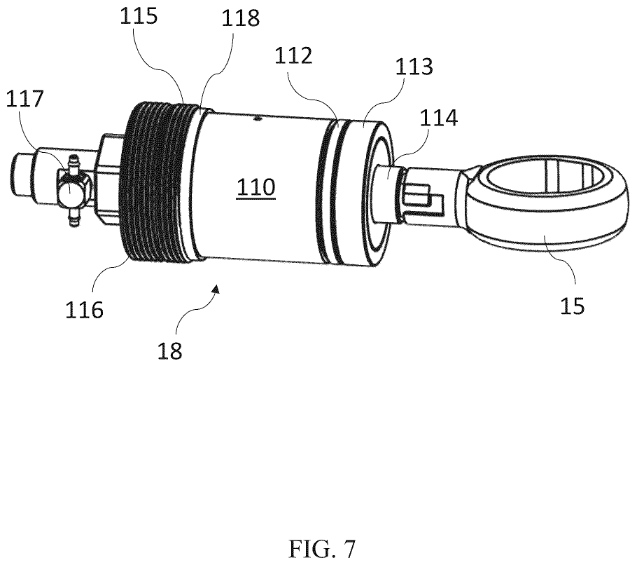

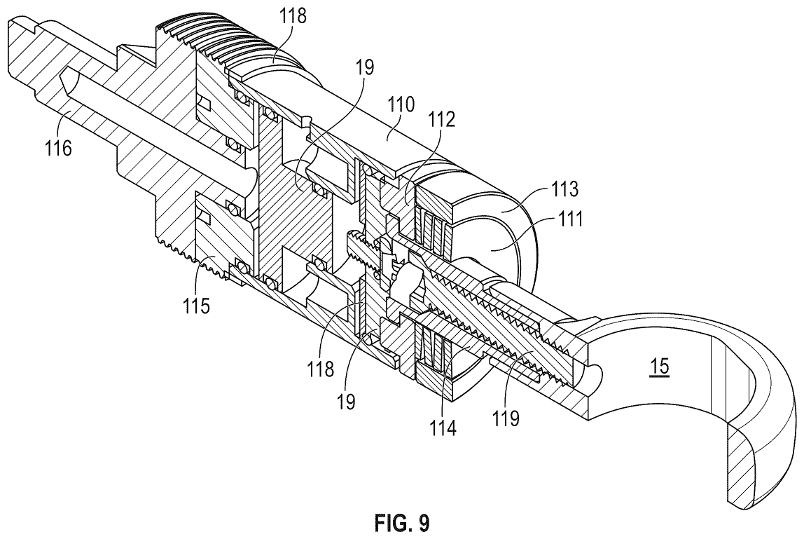

FIGS. 3 through 10 illustrate various views of the distal joint 100 from the mechanical arm 1000 of FIGS. 1 and 2. As illustrated, the distal joint 100 includes a handle 10, a link 11 connecting the handle 10 to a drape connector 130, and a pneumatic assembly 18. Referring briefly to FIG. 4, the handle 10 is connected to a superior end 12 of the link 11 and the drape connector 130 is connected to an inferior end 13 of the link 11. Disposed between the superior end 12 and the inferior end 13 is a ball 14, which is received by and moveable within the pneumatic assembly 18.

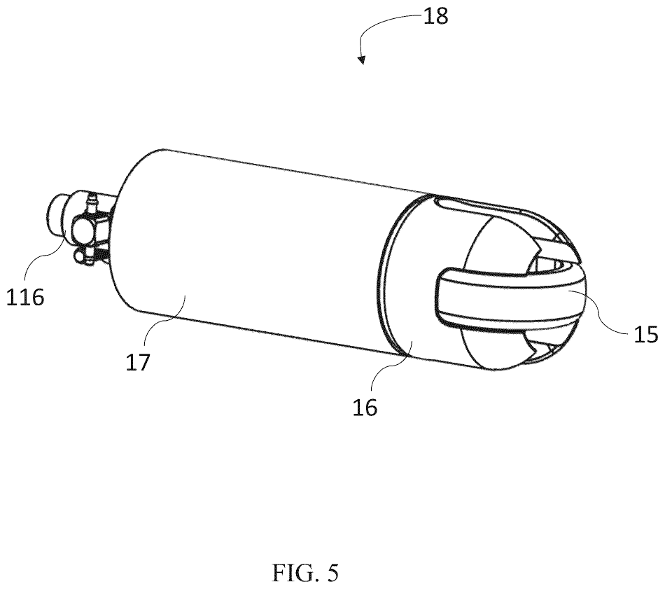

Referring to FIGS. 5 through 10, the pneumatic assembly 18 includes an inner clamp 15, which receives the ball 14 of the link 11, and an outer clamp 16 which receives the inner clamp 15. The pneumatic assembly 18 also includes a housing 17 and a distal joint stem 116. The distal joint stem 116 connects the distal joint 100 to the medial joint 200.

The housing 17 houses and receives a plurality of components of the pneumatic assembly. Specifically, the housing 17 houses an actuator body 110 that holds and receives at least one actuator 19. The housing 17 also houses one or more springs 111 contained within a spring sleeve 113, a spring compressor 112, a spring arbor or anchor 114, and a fluid inlet 115. Further, the housing 17 houses a plurality of seals or gaskets 118, which may be O-rings or spacers.

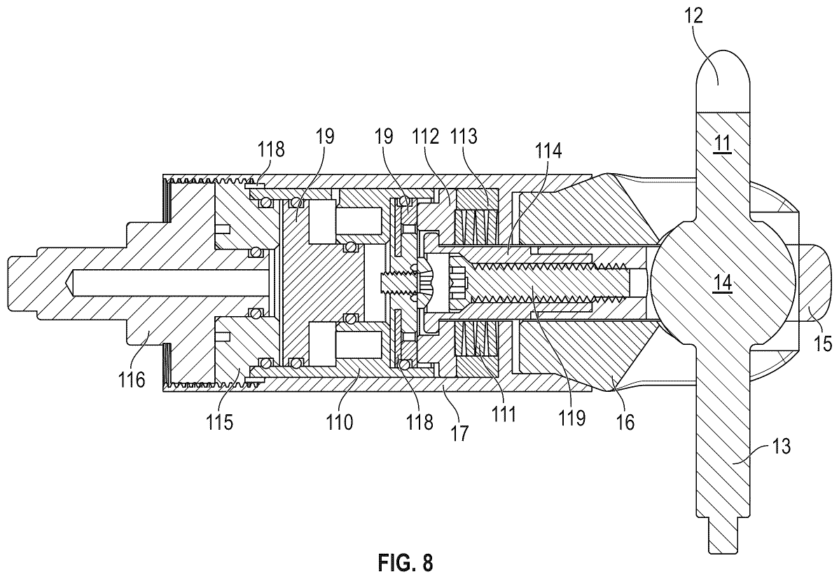

The inner clamp 15 is connected to and rotatable about the spring arbor 114. The inner clamp 15 is also received by the outer clamp 16. Referring briefly to FIG. 8, the ball 14 of the link 11 is seated within the inner clamp 15 and is moveable relative to the inner clamp 15. As illustrated in FIG. 8, the distal joint 100 and/or the pneumatic assembly 18 is closed, such that movement of the inner clamp 15 and the outer clamp 16 relative to the housing 17 is limited. The ball 14 may still rotate relative to the inner clamp 15, but any further motion is prevented.

The distal joint 100 is an air-over-hydraulic joint with a default closed position or closed state. That is, opening of the distal joint 100 by actuation of the at least one actuator 19 is caused by an inlet of fluid (i.e., high-pressure fluid, high-pressure CO₂) into the distal joint 100 through the fluid inlet 115. The fluid inlet 115 may be in communication with a control box loaded with high-pressure CO₂, and in turn, the control box may be in communication with an actuator for activating the high-pressure CO₂ and opening the one or more joints. The actuator may be placed on the handle, or the actuator may be remote from the mechanical arm 1000. When the distal joint 100 receives the high-pressure fluid, the at least one actuator 19 is actuated, causing the spring compressor 112 to compress the springs 111. Compression of the springs 111 causes the outer clamp 16 to mechanically and physically separate from the housing 17, allowing the outer clamp 16 to move relative to the housing 17.

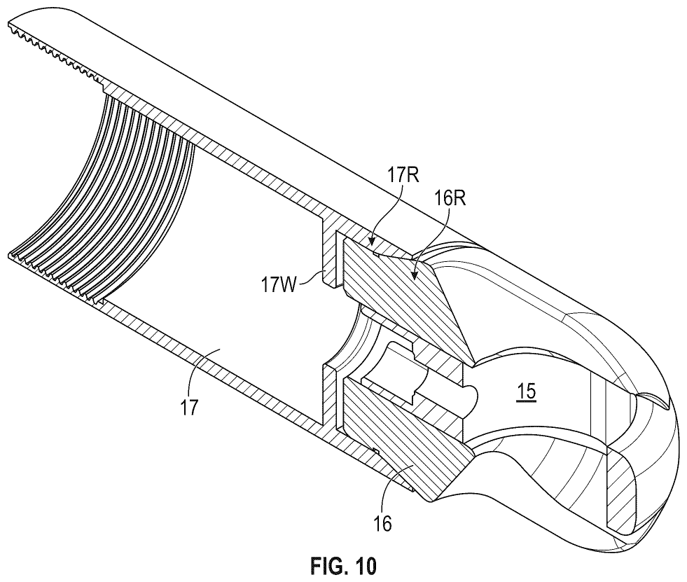

For example, referring briefly to FIG. 10, the housing 17 includes a ramped portion 17R where the housing 17 abuts and receives the outer clamp 16. Similarly, the outer clamp 16 includes a ramped portion 16R. When the distal joint 100 is closed, the ramped portion 17R of the housing 17 abuts the ramped portion 16R of the outer clamp 16. This connection prevents movement of the outer clamp 16 relative to the housing 17. When the distal joint 100 is open, and the springs 111 have been compressed, the ramped portion 16R of the outer clamp 16 is physically separated from the ramped portion 17R of the housing 17. The outer clamp 16 is now free to rotate relative to the housing 17. The springs 111 may include Bellville disk springs or any other appropriate springs. Disc springs are conically-shaped precision components designed to be axially loaded, and Belleville disc springs are capable of large compressive forces.

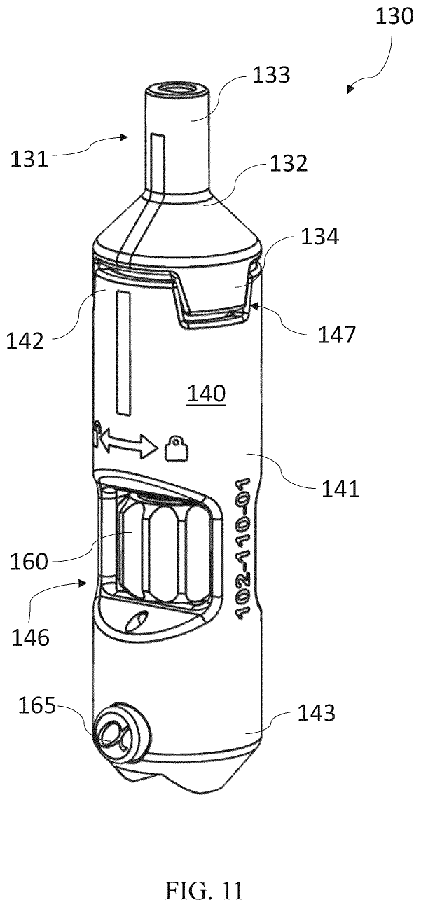

Turning now to the drape connector 130 for connection to the distal joint 100, FIGS. 11 through 16 illustrate the drape connector 130 for attaching a surgical tool to the distal joint 100 across a sterile drape. The drape connector 130 attaches to a connection shank 131 (also referred to herein as “a connection shank”) in association with the mechanical arm 1000. Specifically, the connection shank 131 is associated with or in connection with the inferior end 13 of the link 11. The drape connector 130 attaches to the connection shank 131 across the sterile drape, such that the drape connector 130 is outside the sterile drape.

The drape connector 130 includes a housing 140, a flexible collet 150, and an instrument mounting interface 165. The connection shank 131 includes a body 132 having a first end 133 in connection with the link 11 and a second end 134 in connection with a ball 135 that interfaces with the flexible collet 150 of the housing 140. The ball 135 interfaces with the housing 140 and the flexible collet 150 across the sterile drape without materially tearing or puncturing the sterile drape. If any tears occur in the sterile drape, such tears are contained within the housing 140, maintaining the sterile field for the mechanical arm 1000.

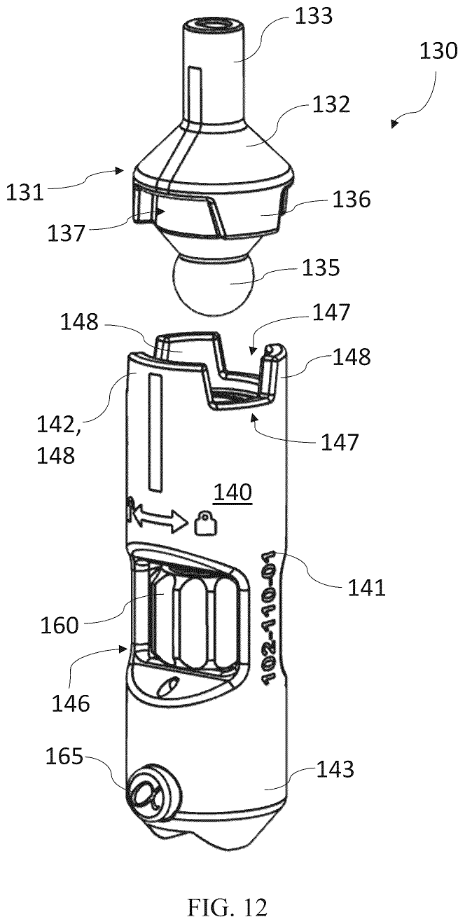

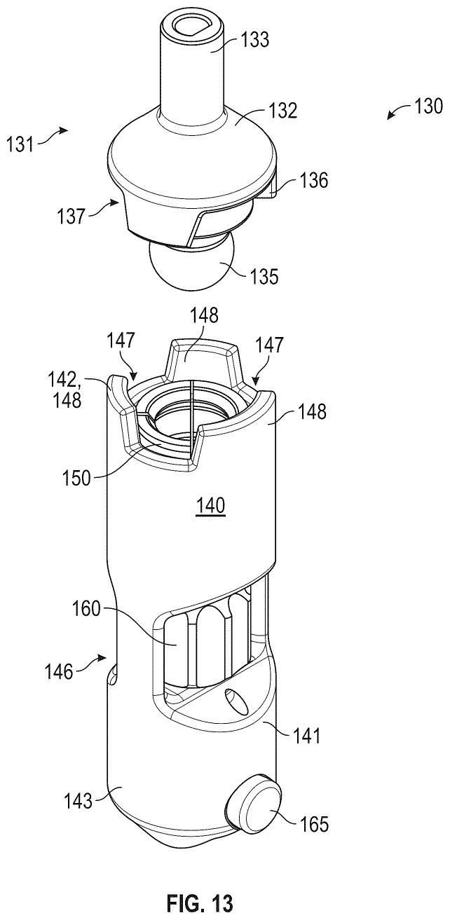

Referring to FIGS. 12 and 13, the body 132 of the connection shank 131 includes a plurality of teeth 136 that define a plurality of gaps 137. The plurality of teeth 136 and the plurality of gaps 137 mechanically engage with a superior end 142 of the housing 140. In some configurations, the teeth may have different sizes to “key” the connection shank 131 to the superior end 142 of the housing 140. The housing 140 includes a body 141 extending between the superior end 142 and an inferior end 143. The superior end 142 includes a plurality of teeth or projections 148 that define a plurality of cut-outs 147. When the connection shank 131 engages the housing 140, the plurality of teeth 136 are received within the plurality of cut-outs 147 and the plurality of projections 148 are received within the plurality of gaps 137. In this way, the connection shank 131 is mechanically engaged with the housing 140. The engagement of the cut-outs and projections creates multiple contact surfaces which limit movement of the connection shank 131 (and therefore movement of the mechanical arm 1000) relative to the housing 140 of the drape connector 130.

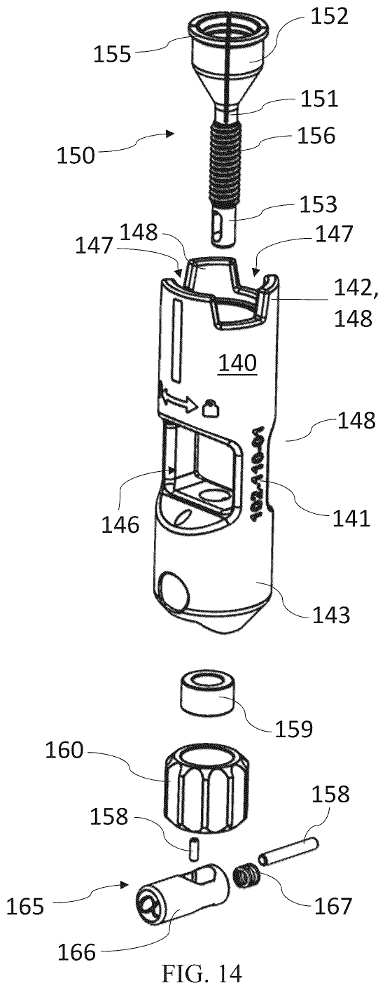

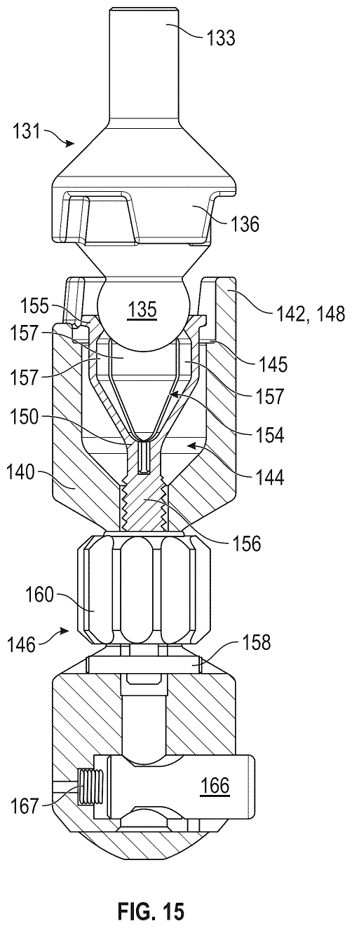

The ball 135 of the connection shank 131 can be positioned adjacent a sterile drape. The ball 135 of the connection shank 131 is received and engaged by the flexible collet 150, which is positioned within the housing 140, across the sterile drape. Referring to FIGS. 14 and 15, the flexible collet 150 has a body 151 extending between a superior end 152 and an inferior end 153. Extending towards the superior end 152 are a plurality of prongs 157 that form a receptacle 154 and a flange 155. The plurality of prongs 157 flex outwardly as the ball 135 is positioned within the receptacle 154 and then snap back around the ball 135 to seat the ball 135 within the receptacle 154.

The flexible collet 150 is adjustable between an unlocked position and a locked position through action of a lock 160 associated with the inferior end 153 of the flexible collet 150. Referring to FIG. 15, the flexible collet 150 is positioned within a channel 144 defined by the body 141 of the housing 140. Disposed near the superior end 142 of the housing 140 is an internal ledge 145. The receptacle 154 and the flange 155, formed by the plurality of prongs 157 of the flexible collet 150, are positioned near the superior end 142 of the housing 140 when the flexible collet 150 is received within the housing 140 in an unlocked position. Once the ball 135 is seated within the receptacle 154, the lock 160 may be actuated to draw the flexible collet inferiorly within the housing 140.

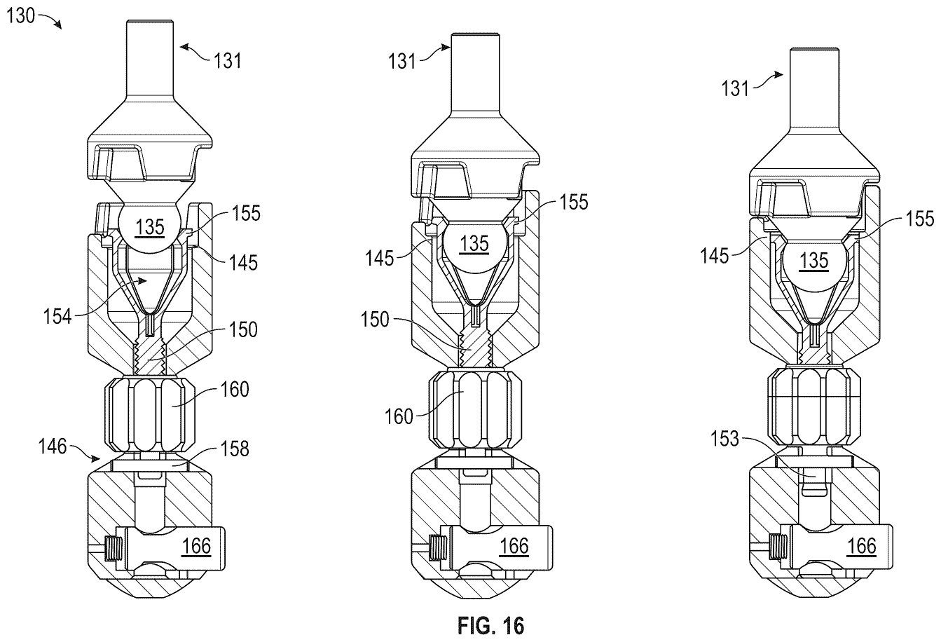

Referring to FIG. 16, actuation of the lock 160 pulls the flexible collet 150 inwardly within the housing 140. As the flexible collet 150 is pulled inwardly, the plurality of prongs 157 move from an extended configuration to a retracted configuration or locked position. Additionally, the flange 155 is pulled past the internal ledge 145 of the housing 140. This tightens the receptacle 154 around the ball 135 and prevents separation of the connection shank 131 (e.g., the ball 135) and the housing 140 (e.g., the flexible collet 150). The flexible collet 150 is anchored within the housing 140 through a dowel pin 158.

The lock 160 is associated with a lock interface 156 of the flexible collet 150. As illustrated, the lock interface 156 includes threads and the lock 160 is a wheel moveable along the threads. The lock 160 is externally accessible through at least one window 146 defined by the body 141 of the housing 140. The lock 160 may be quickly and easily actuated by a practitioner using one hand. In other configurations, the lock 160 may be in connection with the lock interface and located at a different position on the housing 140. For example, the lock may be configured to be actuated by a surgeon's thumb, or rotated by a surgeon's hand, etc. In some embodiments, the lock 160 may extend beyond the edge of the housing 140 or may even be positioned on the outside of the housing 140 for case of access.

A surgical tool or instrument may be connected to the housing 140 through the instrument mounting interface 165. The instrument mounting interface 165 includes a rod 166 for receiving the surgical tool or instrument and a spring 167. The spring 167 biases the rod 166 in a locked configuration. Attachment of the surgical tool to the rod 166 compresses the spring 167, allowing the surgical tool to be connected to the rod 166. Upon connection, the spring 167 will rebound and secure the surgical tool to the inferior end 143 of the housing 140. The surgical tool may now be maneuvered through motions of the distal joint 100 (e.g., rotation, angular adjustment, and forward and backward translation).

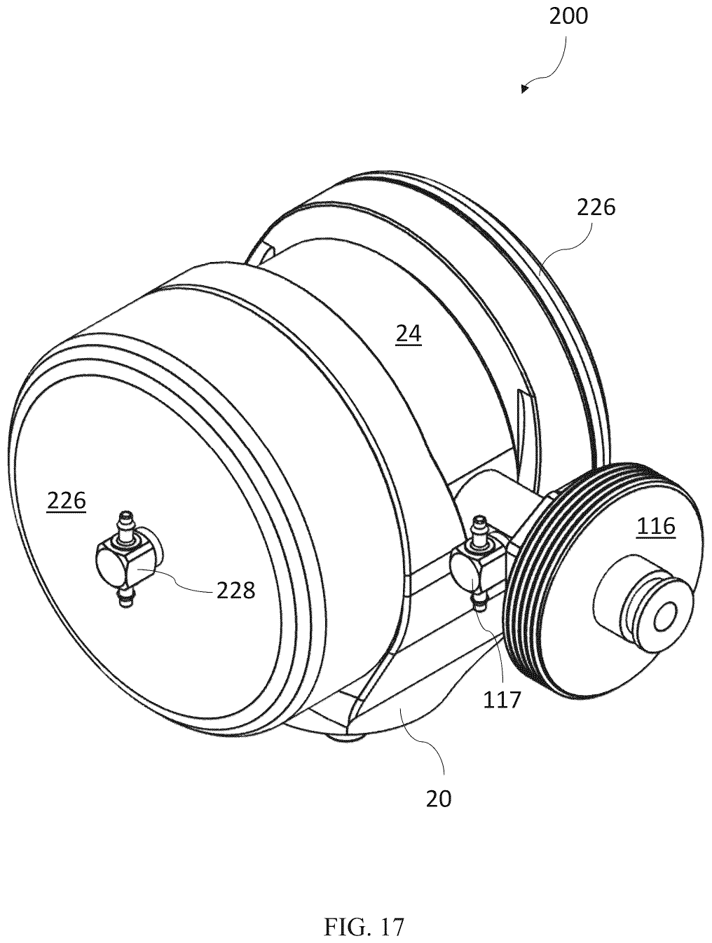

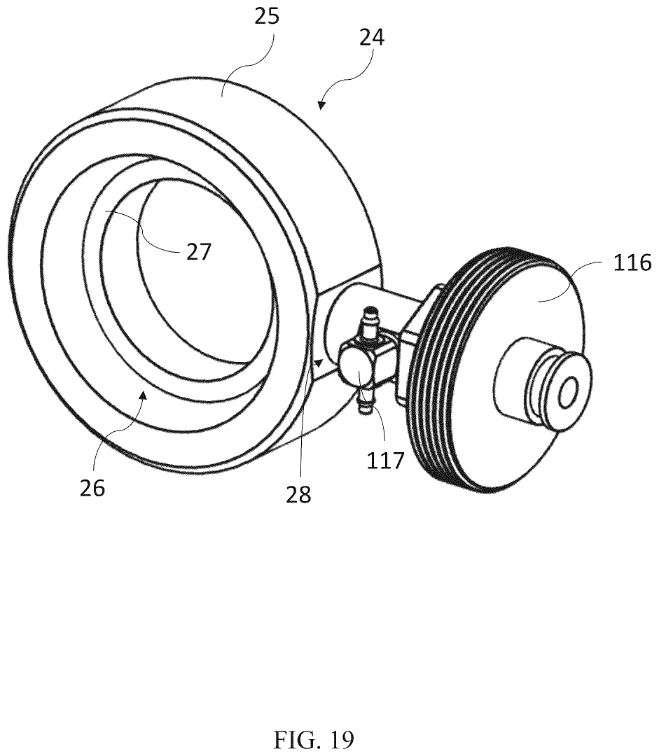

Turning now to the medial joint 200 of the mechanical arm 1000, FIGS. 17 through 21 illustrate the medial joint 200 from the mechanical arm 1000 of FIGS. 1 and 2. The medial joint 200 includes a yoke 20 for receiving a floating or moveable taper flange 24. The yoke 20 also receives caps 226 that house various components. Referring to FIG. 19, the floating taper flange 24 includes a body 25 that is connected to the distal joint stem 116 through a fitting 117. This connection allows the distal joint 100 to pivot about the medial joint 200 when the medial joint 200 is in an open or unlocked configuration.

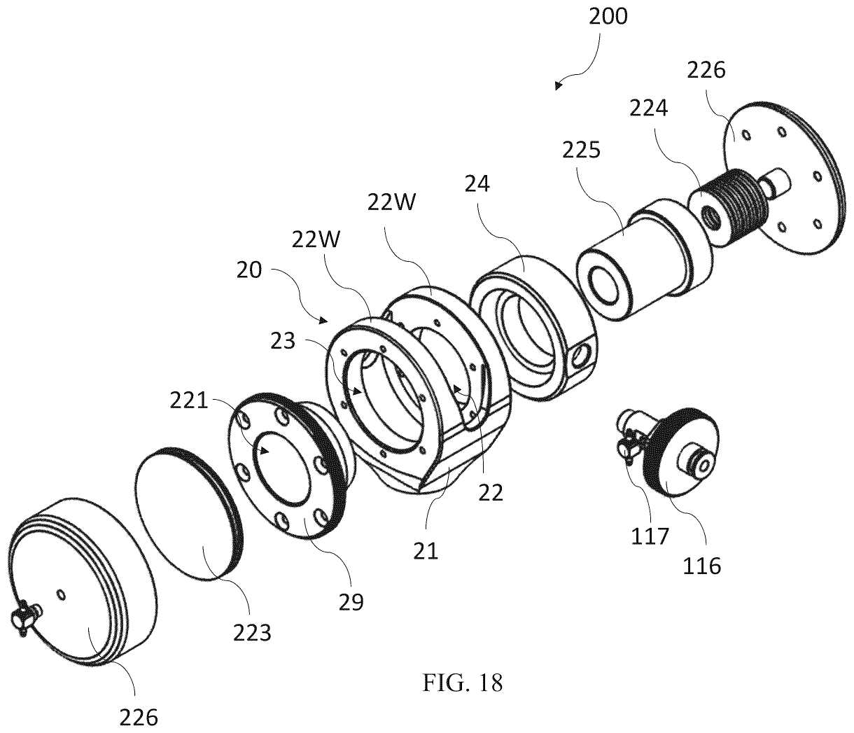

Referring to FIG. 18, the yoke 20 includes a body 21. The body 21 defines a first channel 22 that creates wings 22W. The body 21 and the wings 22W define a second channel 23 that is normal or perpendicular to the first channel 22. The floating taper flange 24 is received within the first channel 22 and is moveable relative to the yoke body 21 when the medial joint 200 is unlocked (i.e., when high-pressure fluid, high-pressure CO₂ is delivered to the mechanical arm 1000). The medial joint 200 also includes a fixed taper 29, an actuator 223, springs 224, and a spring compressor 225. The springs 111 may include Bellville disk springs or any other appropriate springs.

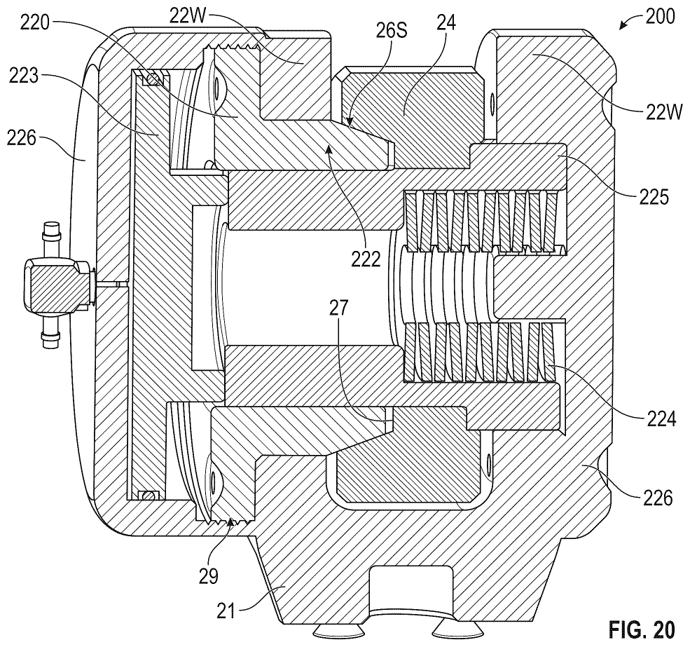

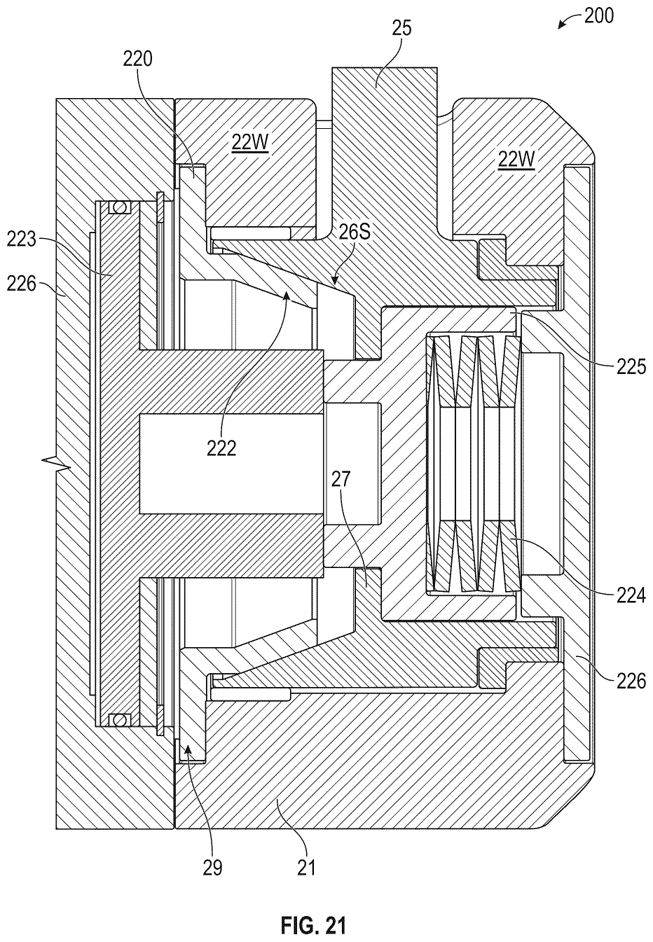

Referring to FIGS. 20 and 21, the floating taper flange 24 is received within the yoke body 21 and mechanically interfaces with the fixed taper 29. Specifically, the floating taper flange 24 defines a cavity 26 that receives and interfaces with a sloped portion 222 of the fixed taper 29. The cavity 26 also receives a portion of the spring compressor 225. The fixed taper 29 also defines a cavity 221 that receives a portion of the spring compressor 225.

The sloped portion 222 abuts a sloped portion 26S and an internal flange 27 of the floating taper flange 24. Similar to the distal joint 100, the physical connection between the sloped portion 222 of the fixed taper 29 and the sloped portion 26S of the floating taper 24 prevents motion of the floating taper 24 relative to the fixed taper 29. The actuator 223 may be a pneumatic actuator 223 that is moveable upon introduction of a high-pressure fluid into the medial joint 200.

Specifically, actuation of the actuator 223 causes the spring compressor 225 to compress the springs 224. Compression of the springs 224 allows the ramped portion 26S of the floating taper flange 24 to separate from the ramped portion 222 of the fixed taper 29. This separation allows the floating taper flange 24 to move relative to the fixed taper 29. As the floating taper flange 24 is connected to the distal joint 100, movement of the floating taper flange 24 relative to the fixed taper 29 also moves the distal joint 100. In this way, the distal joint 100 may be pivoted about the medial joint 200. In other words, compression of springs 224 disengages the fixed/floating taper, but does not force the tapers to open but rather creates a gap. This relatively small taper separation allows movement of the joints, while reducing the perception of drift between the open and closed states.

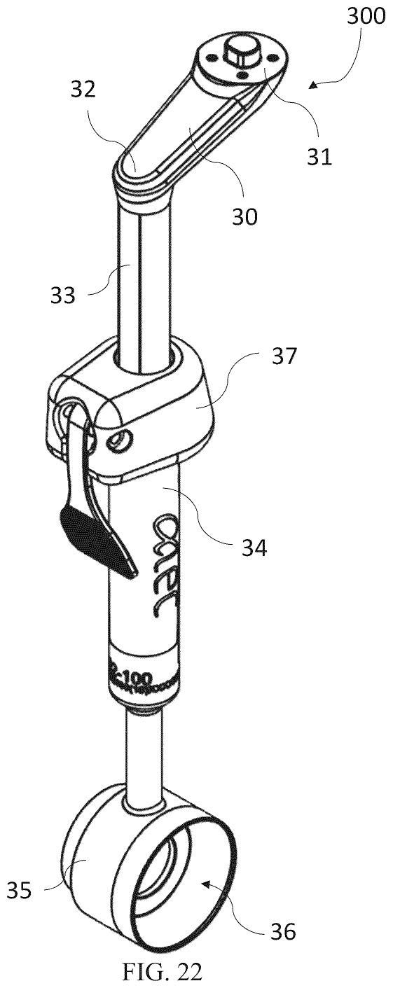

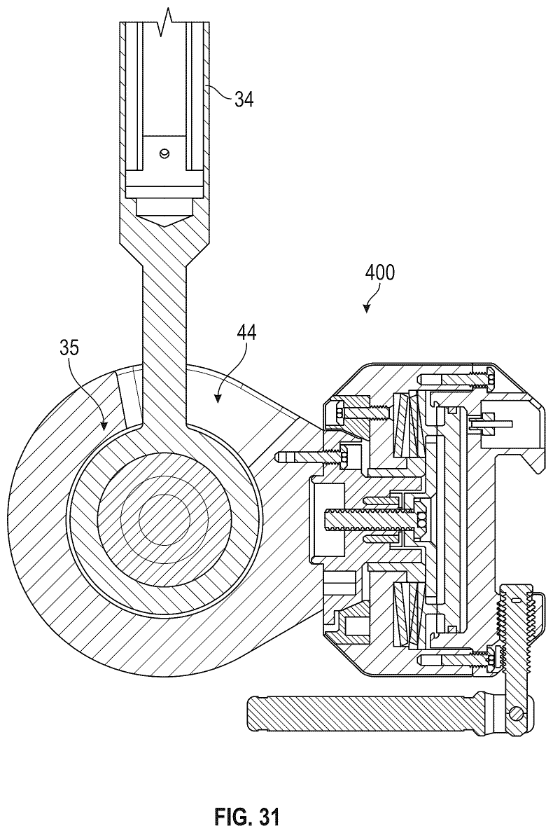

FIGS. 22 through 24 illustrate the translating link 300 from the mechanical arm 1000 of FIGS. 1 and 2. As illustrated, the translating link 300 includes an adaptor 30 for attaching the translating link 300 to the yoke body 21 of the medial joint 200. Specifically, a superior end 31 attaches to the yoke body 21 and an inferior end 32 of the adaptor 30 is in connection with a tube or rod 33. The tube or rod 33 is adjustable relative to an axle 34 through action of a lock 37. A receiver 35 is positioned at an inferior end of the axle 34 and defines a cavity 36 to receive at least a portion of the proximal joint 400. FIG. 31 illustrates a cross-sectional view of the proximal joint 400 in connection with the receiver 35.

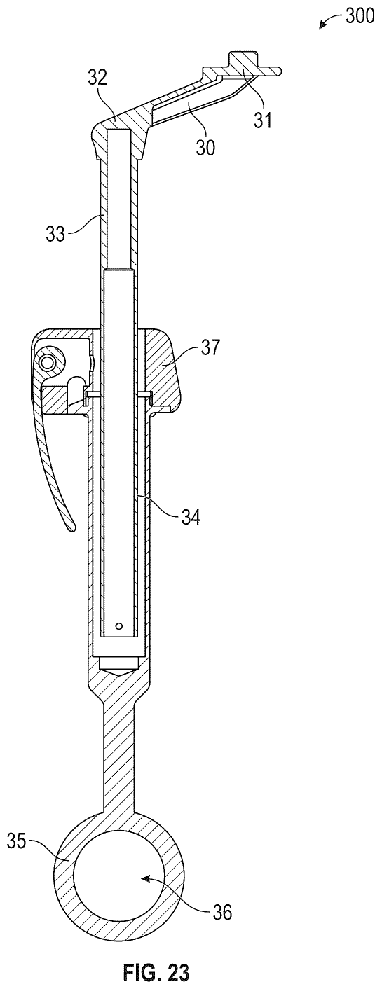

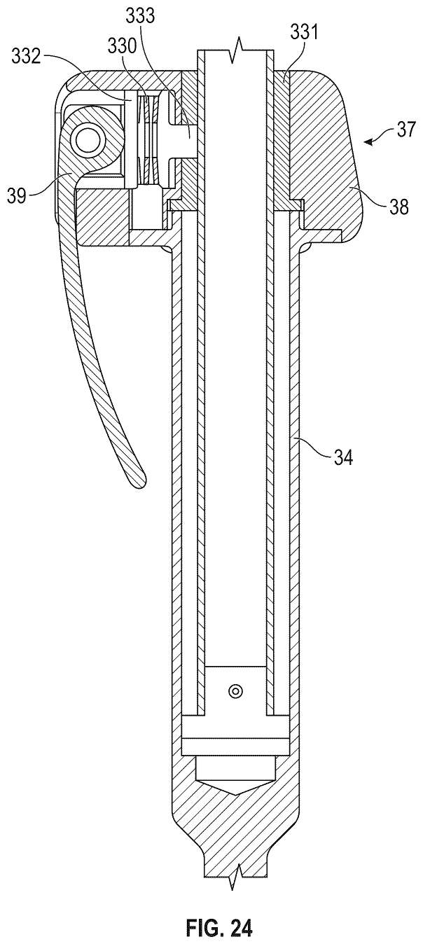

Specifically, referring to FIGS. 23 and 24, the rod 33 is nestable or telescope-able within the axle 34. Actuation of the lock 37 unlocks the translating link 300 and allows the rod 33 to be adjusted up or down relative to the axle 34, thereby lengthening or shortening the mechanical arm 1000. The lock 37 may be a cam lock or any other suitable lock. The lock 37 may include a housing 38 that receives a spring 330, a spring compressor 332, and a clamp 333. The lock 37 may also include a bushing 331 positioned within the housing 38 and around the axle 34, and a lever 39. The lever 39 may selectively compress the springs 330, through compression of the spring compressor 332, to close the lock 37.

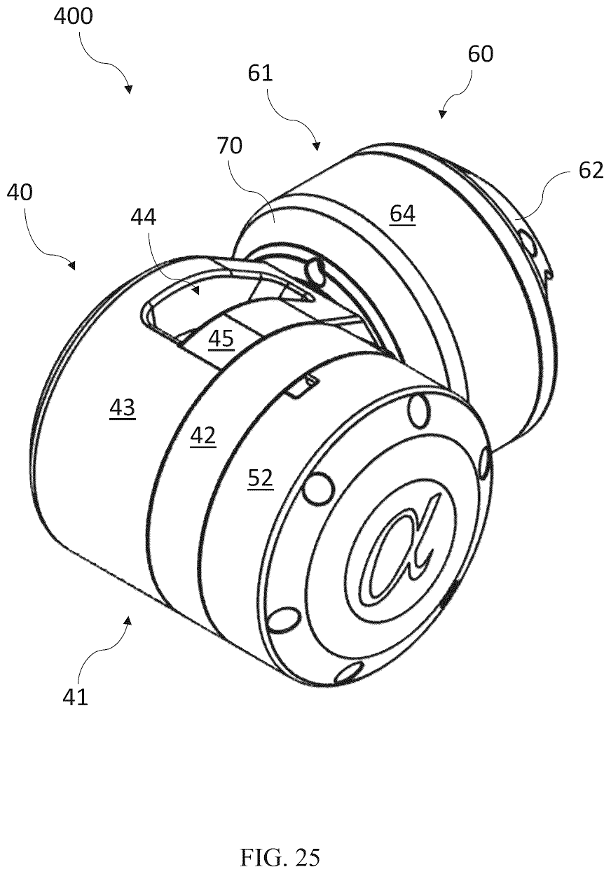

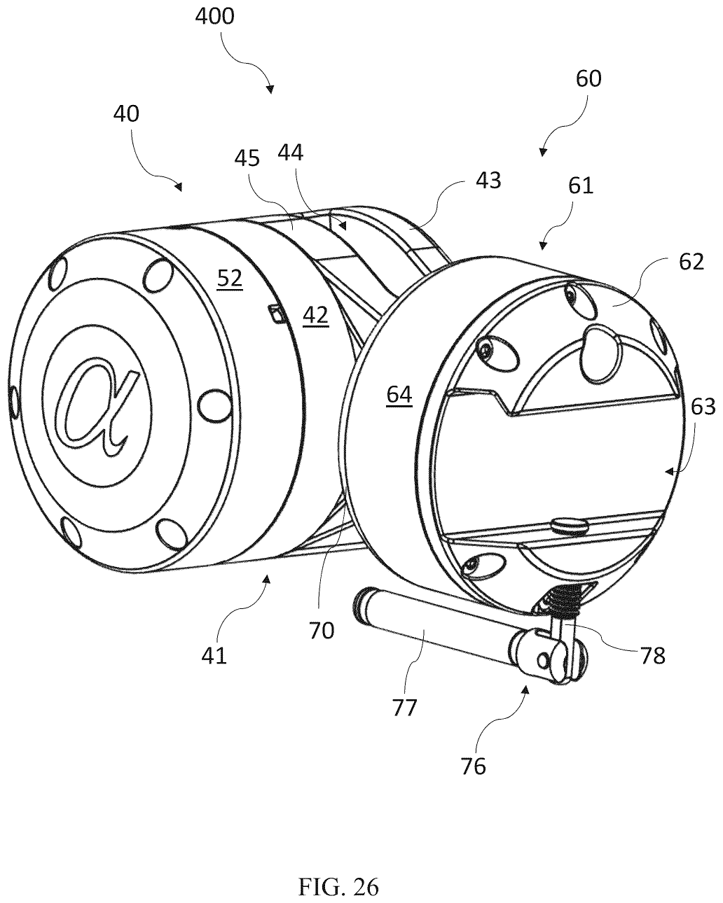

FIGS. 25 through 31 illustrate the proximal joint 400 from the mechanical arm 1000 of FIGS. 1 and 2. The proximal joint 400 is formed from two joints—a medial-lateral joint 40 and a cranial-caudal joint 60. The medial-lateral joint 40 connects to the translating link 300 via the receiver 35 and allows the mechanical arm 1000 to be moved forward and backward. The cranial-caudal joint 60 secures the mechanical arm 1000 to a surgical table and allows the mechanical arm 1000 to pivot about the cranial-caudal joint 60.

The medial-lateral joint 40 includes a housing 41 that has a front plate 42 and a back plate 43. The front plate 42 receives a cap 52. The back plate 43 defines a slot 44 through which a portion of the axle 34 of the translating link 300 extends. The back plate 43 also includes a keystone 45, which allows for assembly of the translating link 300 to the medial-lateral joint 40.

The cranial-caudal joint 60 similarly includes a housing 61 that has a front plate 62 and a back plate 64. The front plate 62 may define a slot or channel 63 that allows the proximal joint 400 to be secured to a surgical table. The cranial-caudal joint 60 also includes a clamping mechanism 76 for securing the proximal joint 400 to the surgical table.

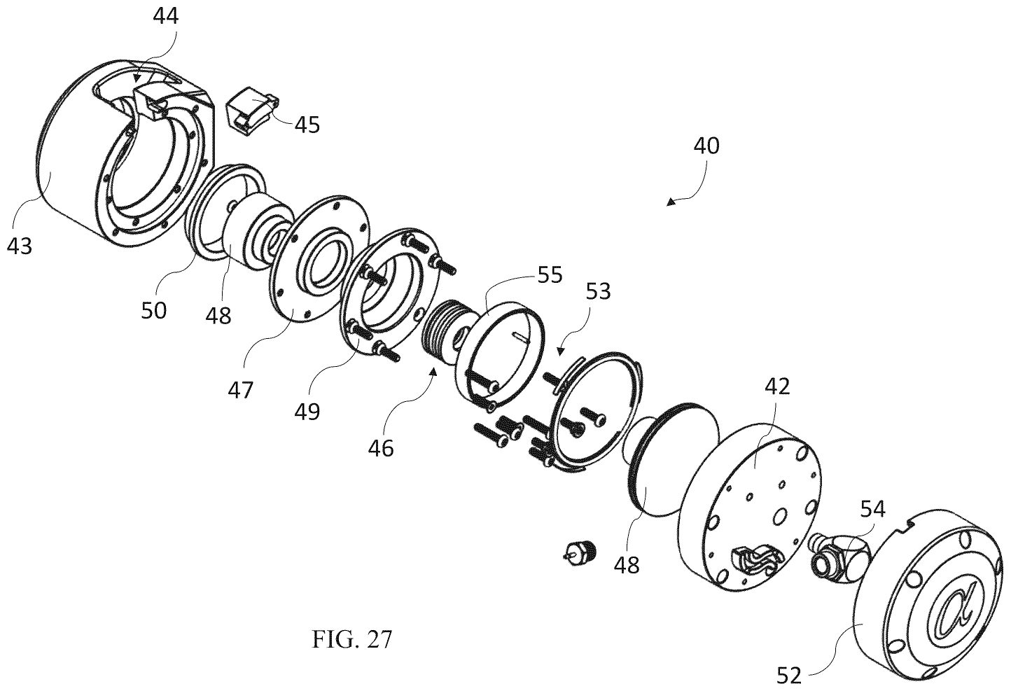

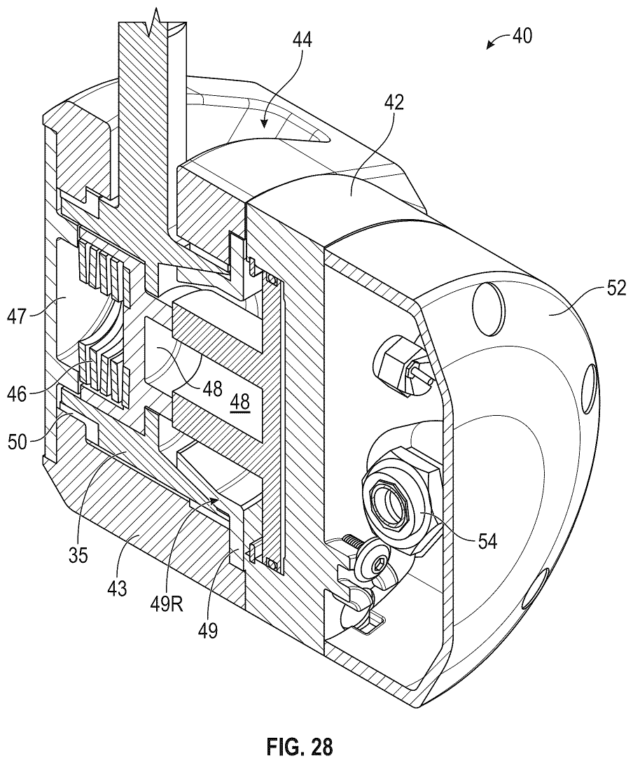

FIG. 27 illustrates an exploded view of the medial-lateral joint 40 and FIG. 28 illustrates a cross-sectional view of the medial-lateral joint 40 in connection with the receiver 35 of the translating link. As illustrated, the medial-lateral joint 40 includes the housing 41 with the front plate 42 and the back plate 43. Housed between the front plate 42 and the back plate 43 is at least one actuator 48 (e.g., two actuators 48), springs 46, an inner taper 49, and a flanged bearing 50. A spring compressor 47 is in association with the back plate 43.

Disposed between the front plate 42 and the cap 52 is a valve 54 that delivers high-pressure fluid to the actuator(s) 48. Upon delivery of the high-pressure fluid, the actuator(s) 48 compress the springs 46 against the spring compressor 47. Similar to the distal joint 100 and the medial joint 200, compression of the springs 46 causes a separation between the inner taper 49 and the receiver 35 of the translating link. Specifically, a ramped portion 49R of the inner taper 49 abuts the cavity 36 of the receiver 35 when the medial-lateral joint 40 is closed or locked. Compression of the springs 46 causes a separation between the ramped portion 49R of the inner taper 49 and the cavity of the receiver 35, allowing the translating link 300 to be moved relative to the medial-lateral joint 40 within the slot 44.

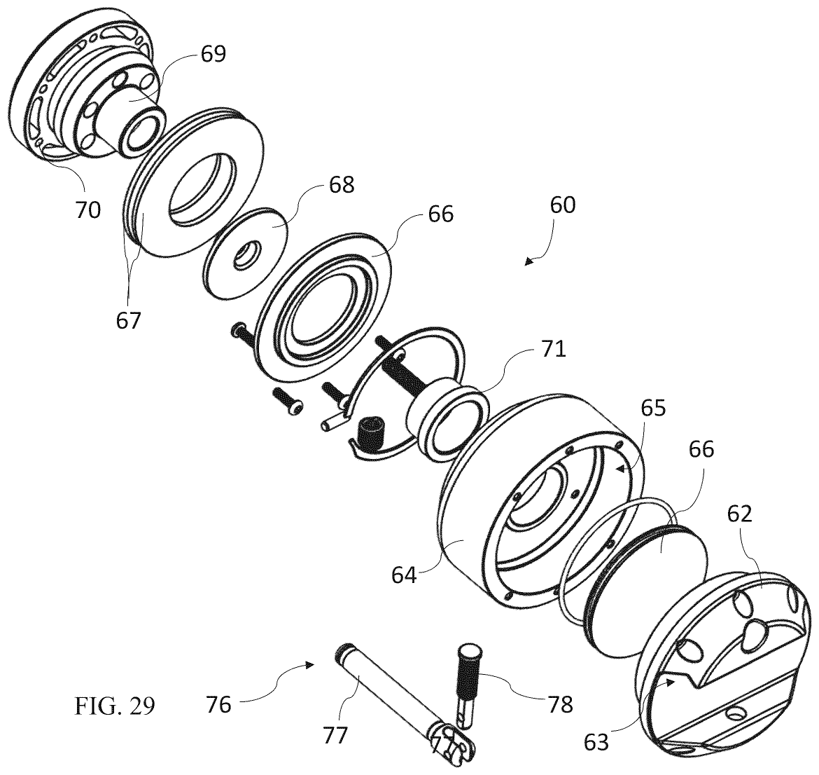

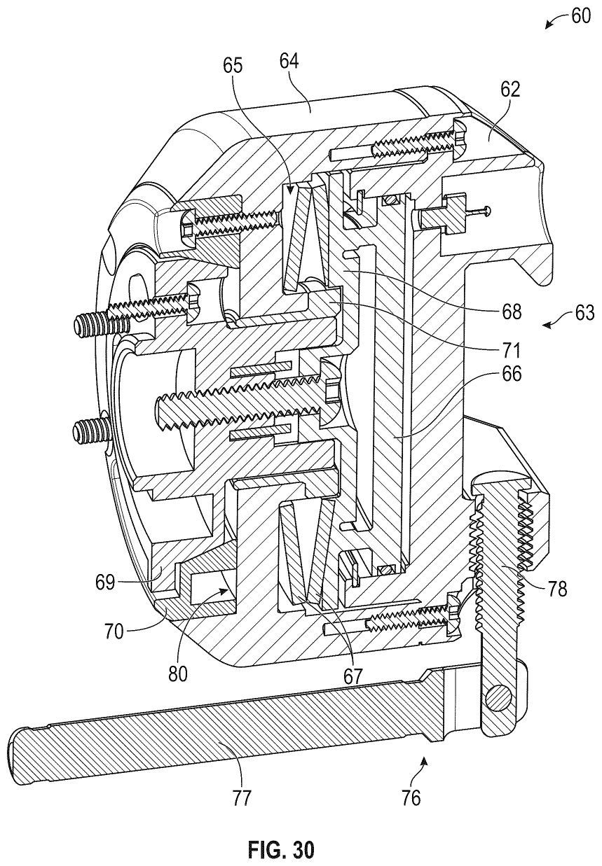

FIG. 29 illustrates an exploded view of the cranial-caudal joint 60 and FIG. 30 illustrates a cross-sectional view of the cranial-caudal joint 60. As illustrated, the cranial-caudal joint 60 includes the housing 61 having a front plate 62 and a back plate 64. The front plate 62 defines the slot 63 for connecting the cranial-caudal joint 60 to a surgical table. The back plate 64 defines a first cavity 65 for receiving at least one actuator 66, springs 67, and a spring compressor 68. The back plate 64 also defines a second cavity 80 for receiving an outer taper 70 and an inner taper 69.

Similar to the medial-lateral joint 40, the distal joint 100, and the medial joint 200, compression of the springs 67 by the spring compressor 68 upon actuation of the actuator(s) 66 causes the inner taper 69 to separate from the outer taper 70. Upon separation, the inner taper 69 may rotate relative to the outer taper 70, allowing the mechanical arm 1000 to be rotated. Specifically, the inner taper 69 is connected to the medial-lateral joint 40 through a plurality of fasteners 75. Rotation of the inner taper 69 allows for rotation of the medial-lateral joint 40 and, thus, rotation of the mechanical arm 1000.

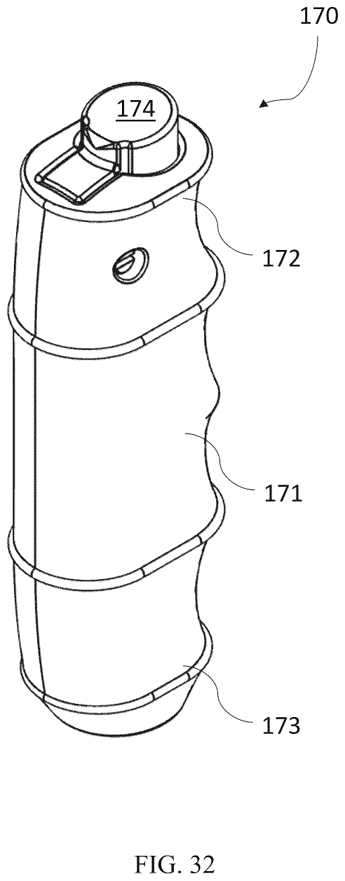

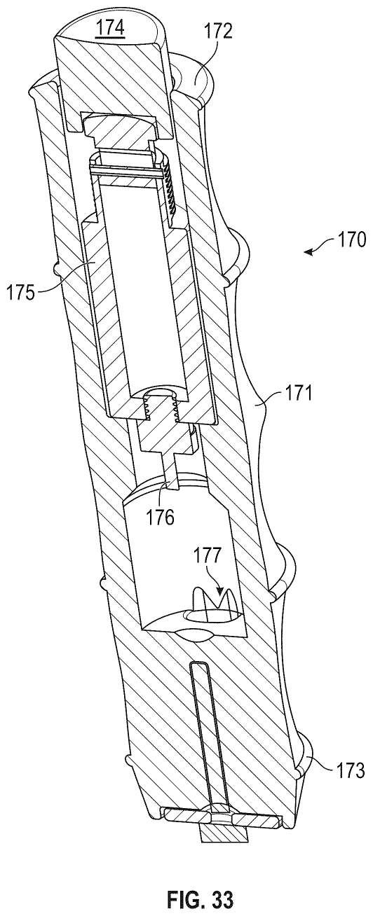

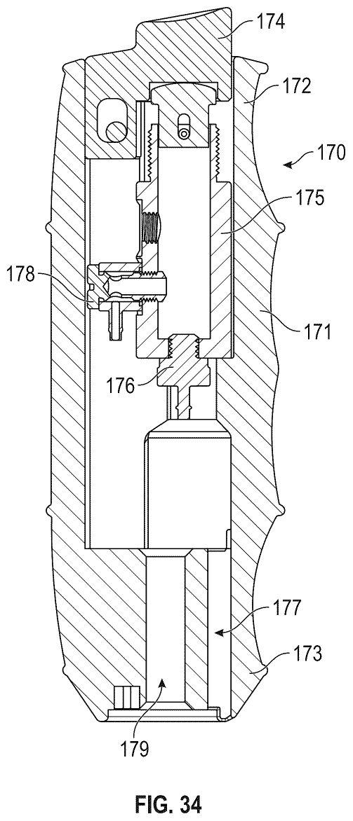

FIGS. 32 through 35 illustrate the handle 170 from the distal joint 100 and the mechanical arm 1000. The handle 170 includes a body 171 that extends between a superior end 172 and an inferior end 173. The inferior end 173 defines a channel 179 to receive the superior end 12 of the link 11 and a channel 177 for connection to a high-pressure fluid line. Disposed at the superior end 172 is an actuator or button 174 for introducing a high-pressure fluid into the mechanical arm 1000. The button 174 is in communication with an air valve 175 and a barb fitting 176, where the barb fitting 176 is for connection to the high-pressure fluid line. A fitting 178 secures the air valve 175 within an interior of the handle body 171 (see FIG. 34). The handle 170 with the actuator or button 174 can be directly connected to the mechanical arm 1000, or it may be remote from the arm, as desired and depending on the surgical application for the mechanical arm 1000.

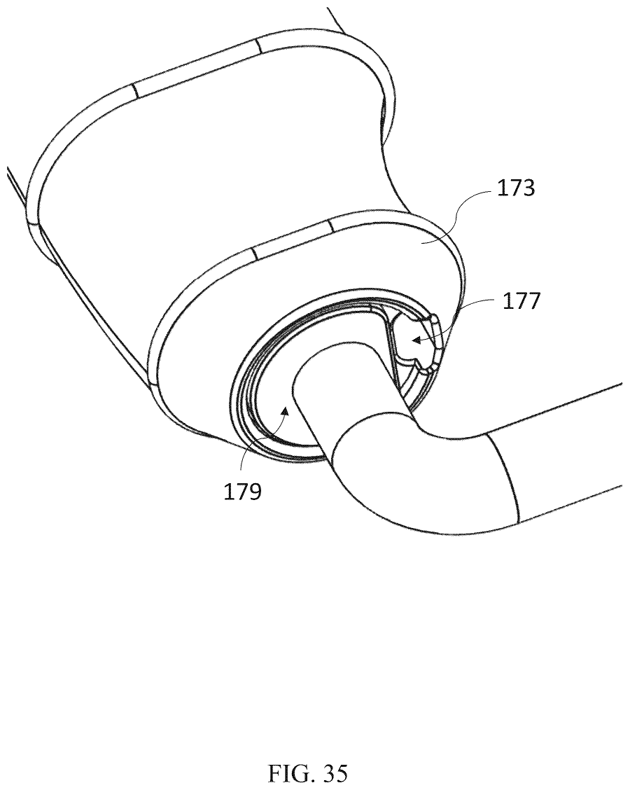

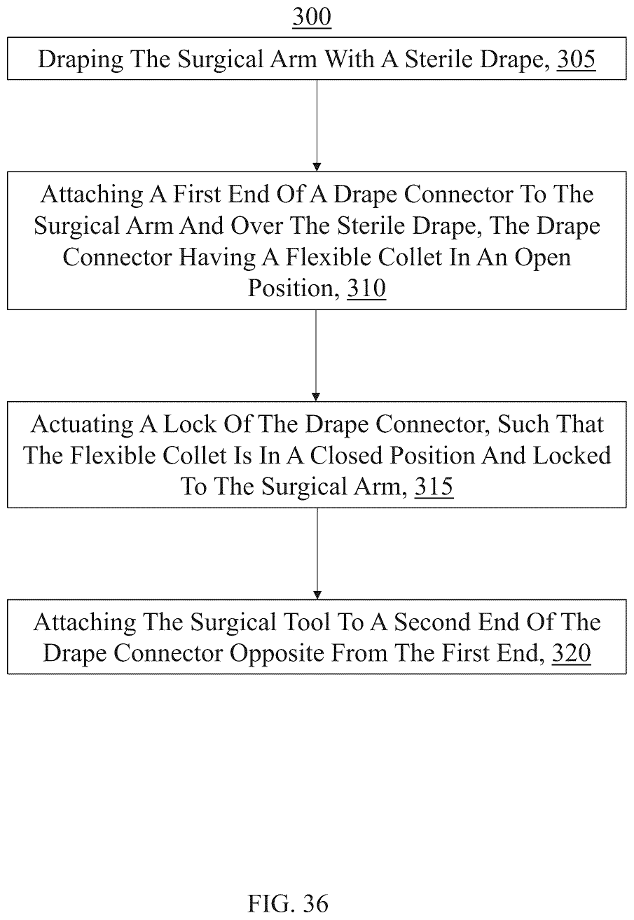

FIGS. 36 through 38 are flowcharts of example methods according to the present disclosure. FIG. 36 is a flowchart of a method 300 of connecting a surgical tool to a surgical arm, such as the mechanical arm 1000. The method 300 includes draping the surgical arm with a sterile drape, at 305. The sterile drape may cover a distal joint 100, a medial joint 200, a translating link 300, and a proximal joint 400 of the mechanical arm 1000. The method 300 may also include attaching a first end of a drape connector to the surgical arm and over the sterile drape, the drape connector having a flexible collet in an open position, at 310. The drape connector may be the drape connector 130 of FIGS. 11 through 16. The method 300 may further include actuating a lock of the drape connector, such that the flexible collet is in a closed position and locked to the surgical arm, at 315. Additionally, the method 300 may include attaching the surgical tool to a second end of the drape connector opposite from the first end, at 320.

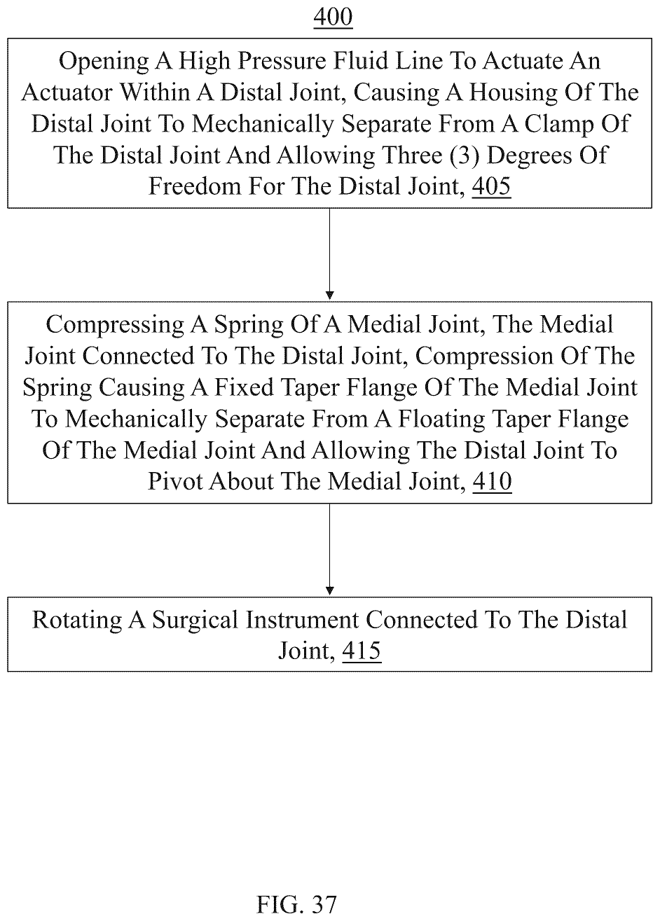

FIG. 37 is a flowchart of a method 400 of using a mechanical surgical arm, such as the mechanical arm 1000. The method 400 may include opening a high pressure fluid line to actuate an actuator within a distal joint, causing a housing of the distal joint to mechanically separate from a clamp of the distal joint and allowing three (3) degrees of freedom for the distal joint, at 405. The method 400 may also include compressing a spring of a medial joint, the medial joint connected to the distal joint, compression of the spring causing a fixed taper flange of the medial joint to mechanically separate from a floating taper flange 24 of the medial joint and allowing the distal joint to pivot about the medial joint, at 410. Further, the method 400 may include rotating a surgical instrument connected to the distal joint, at 415.

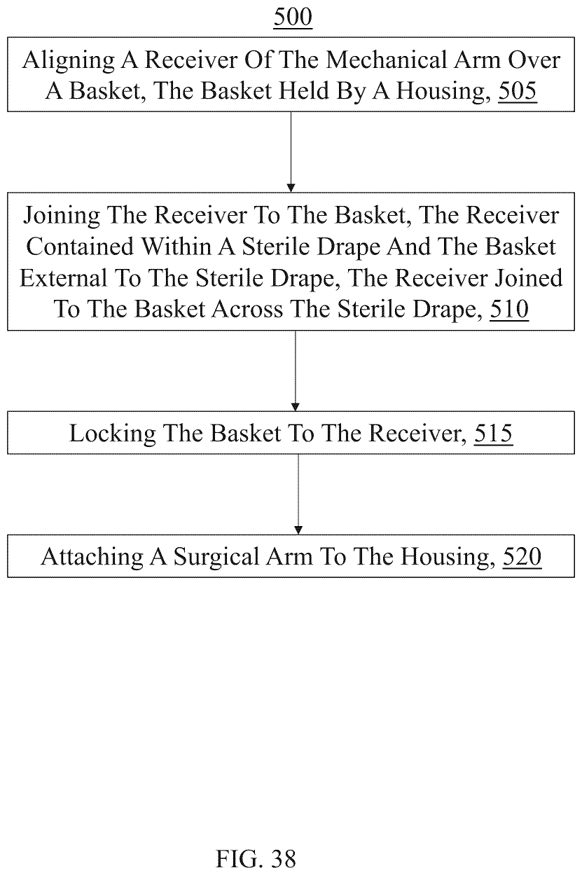

FIG. 38 is a flowchart of a method 500 of attaching a surgical instrument to a mechanical arm, such as the mechanical arm 1000. The method 500 may include aligning a receiver of the mechanical arm over a basket, the basket held by a housing, at 505. The method 500 may also include joining the receiver to the basket, the receiver contained within a sterile drape and the basket external to the sterile drape, the receiver joined to the basket across the sterile drape, at 510. The method 500 may further include locking the basket to the receiver, at 515, and attaching a surgical tool (e.g., a retractor, blade assembly, etc.) to the housing, at 520.

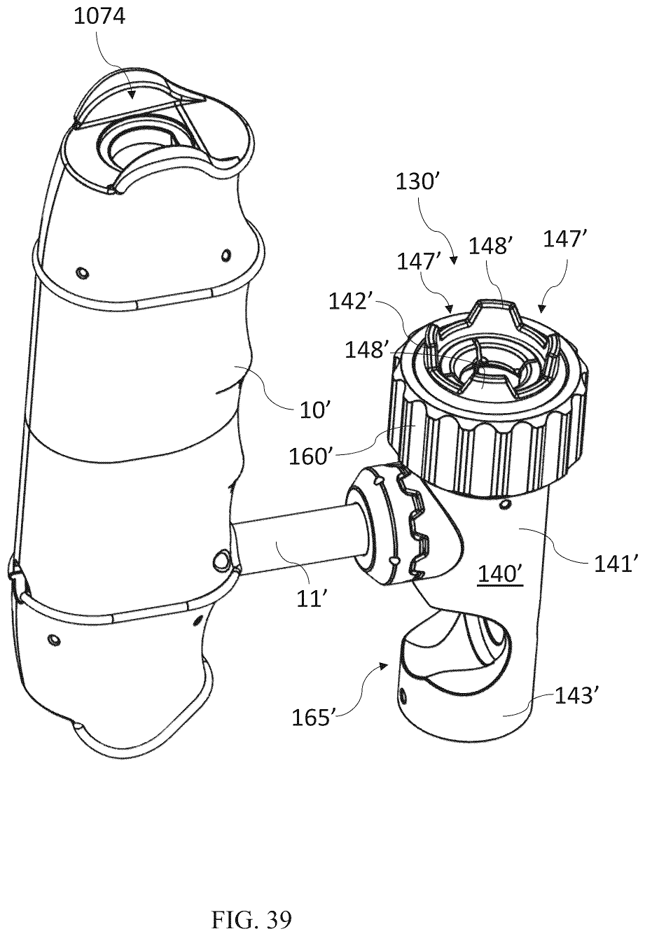

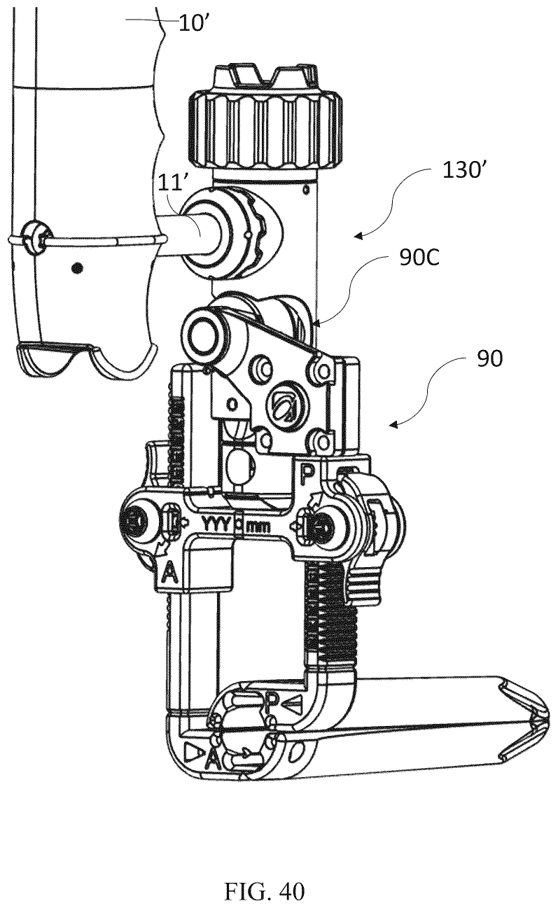

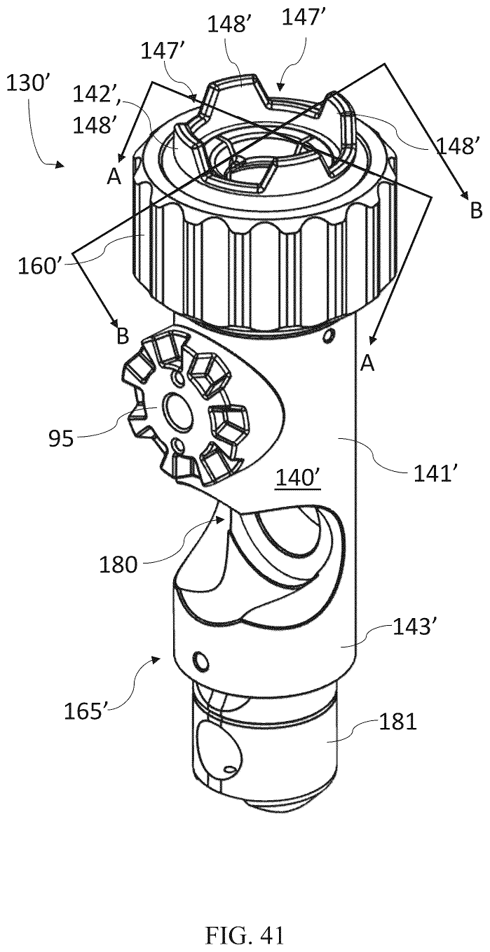

FIGS. 39 through 43 illustrate another embodiment of a handle 10′ and drape connector 130′ for connecting a surgical instrument to the mechanical arm 1000 of FIGS. 1 and 2. As the handle 10′ and the drape connector 130′ share characteristics of the handle 10 and the drape connector 130 of FIGS. 1 through 35, like elements will be labelled with like reference numbers. The handle 10′ is connectable directly to the drape connector 130′ through a link 11′. The link 11′ engages with a control mechanism 95 that facilitates movement of the drape connector 130′ (and a surgical instrument 90 attached to the drape connector 130′) within a surgical process. The handle 11′ is securable to the control mechanism 95 through a fastener (e.g., a screw, rod, etc., not illustrated).

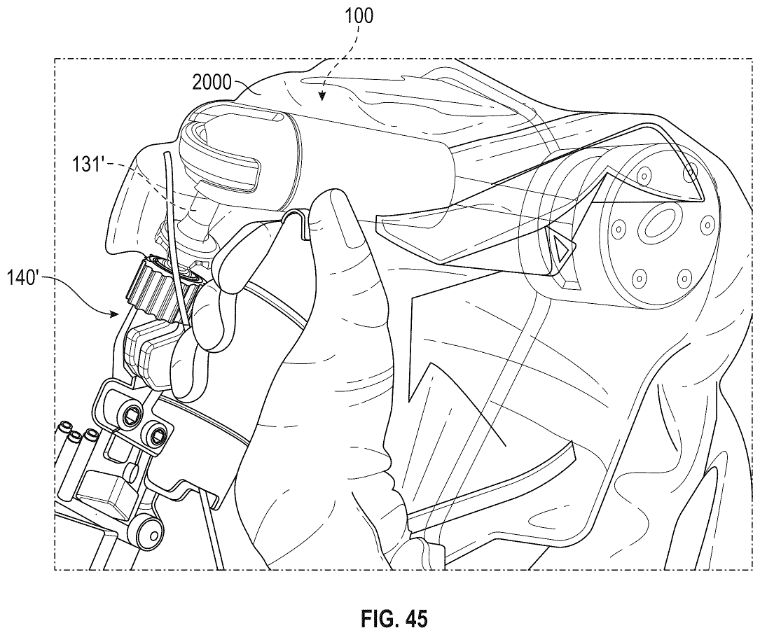

The drape connector 130′ includes a housing 140′ having a body 141′ that extends between a superior end 142′ and an inferior end 143′. Similar to the drape connector 130 of FIGS. 11 through 16, the superior end 142′ includes a plurality of teeth or projections 148′ defining a plurality of cut-outs 147′. The plurality of projections 148′ and cut-outs 147′ mechanically engage with a plurality of teeth 136′ and a plurality of gaps 137′ of a connection shank 131′ (see, for example, the connection across the sterile drape 2000 in FIG. 45).

The inferior end 143′ of the housing 140′ includes an instrument mounting interface 165′. The instrument mounting interface 165′ includes a window or cavity 180 and an adaptor 181. The window 180 may receive and engage a connector 90C of the surgical tool 90 and the adaptor 181 may be secured to a portion of the surgical tool 90.

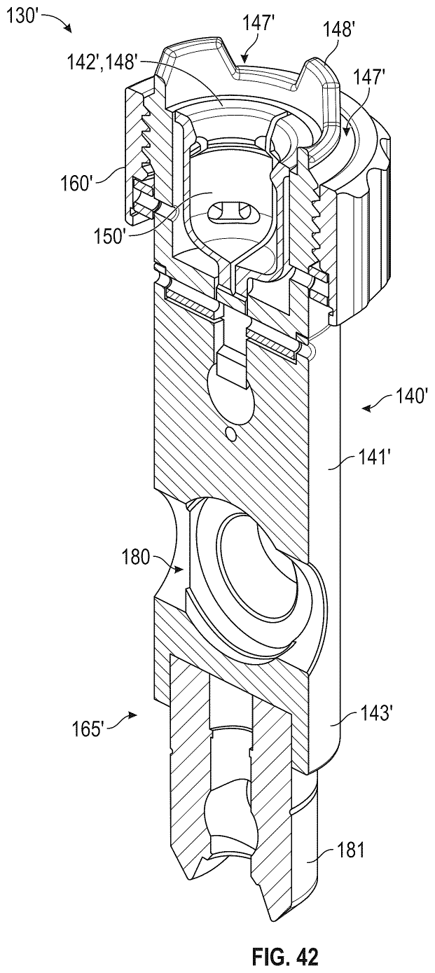

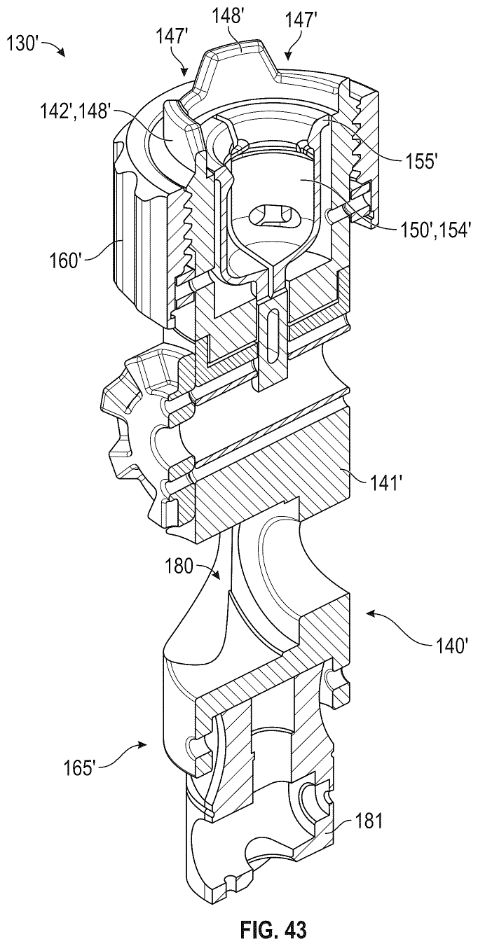

As before, the housing 140′ receives and contains a flexible collet 150′ within an interior of the housing 140′. The flexible collet 150′ may be movable from an extended and unlocked position to a retracted and locked position within the housing 140′. As illustrated in FIGS. 42 and 43, the flexible collet 150′ is in the retracted and locked position. A lock 160′ can be actuated to move the flexible collet 150′ from the extended position to the retracted position. For example, the lock 160′ may be a thumbwheel and actuation of the lock 160′ in a first direction causes the flexible collet 150′ to move from the extended position to the retracted position. As illustrated, the lock 160′ may be a threaded thumbwheel engaged by threads at the superior end 142′ of the housing 140′. Retraction of the flexible collet 150′ within the housing 140′ causes the flange 155′ to be pulled over an internal ledge 145′ of the housing 140′, thereby securing the flexible collet 150′ in its retracted and locked position.

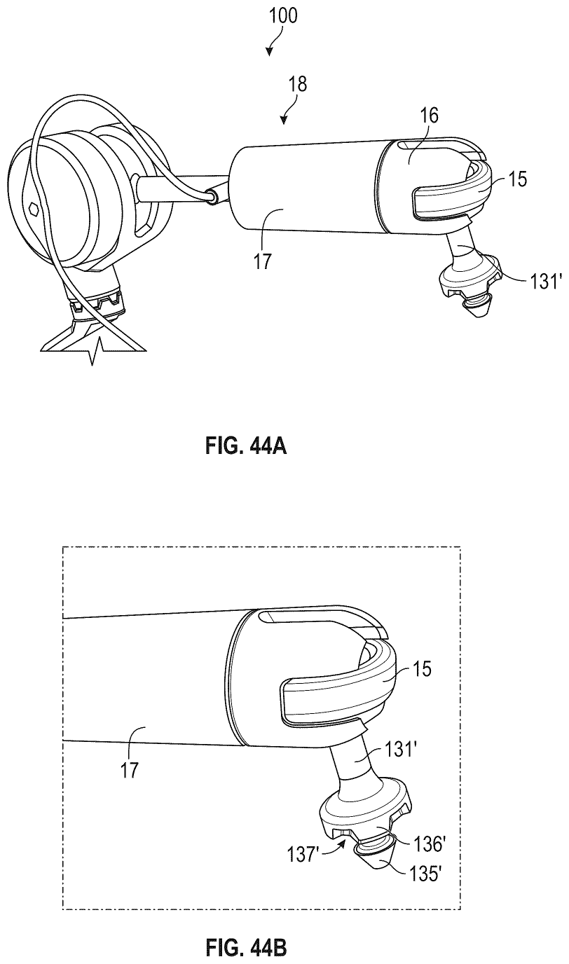

Referring briefly to FIGS. 44A through 45, the drape connector 130′ is connectable to the connection shank 131′, thereby connecting the drape connector 130′ with the distal joint 100 of the mechanical arm 1000 across a sterile drape 2000. When the drape connector 130′ is connected to the connection shank 131′, the handle 10′ remains outside of the sterile drape 2000. The connection shank 131′ includes a first ball joint 135′ that is connectable to the drape connector 130′. Similar to connection shank 131 and drape connector 130, the ball joint 135′ is received within a flexible collet 150′ that is contained within the housing 140′ of the drape connector 130′. As before, retraction of the flexible collet 150′ within the housing 140′ (through actuation of the lock 160′) causes the flexible collet 150′ to lock around the ball joint 135′. Specifically, the receptacle 154′ of the flexible collet 150′ retracts around the ball joint 135′.

The connection shank 131′ also includes a second ball joint (not illustrated) that is received within the inner clamp 15 of the distal joint 100. The second ball joint may move freely within the inner clamp 15. As before, the inner clamp 15 is movable relative to the housing 17 of the pneumatic assembly 18 when the pneumatic assembly 18 is actuated upon introduction of high-pressure fluid into the pneumatic assembly 18. Upon actuation of an actuator 1074 included within the handle 10′, high-pressure fluid is introduced into the pneumatic assembly 18, causing an actuator (e.g., actuator 19, a pneumatic piston, etc.) to compress the spring compressor 112, which compresses the springs 111 (see FIG. 8). Compression of the springs 111 causes the outer clamp 16 to mechanically and physically separate from the housing 17, allowing the outer clamp 16 to move relative to the housing 17 (e.g., rotate, angular movement, etc.).

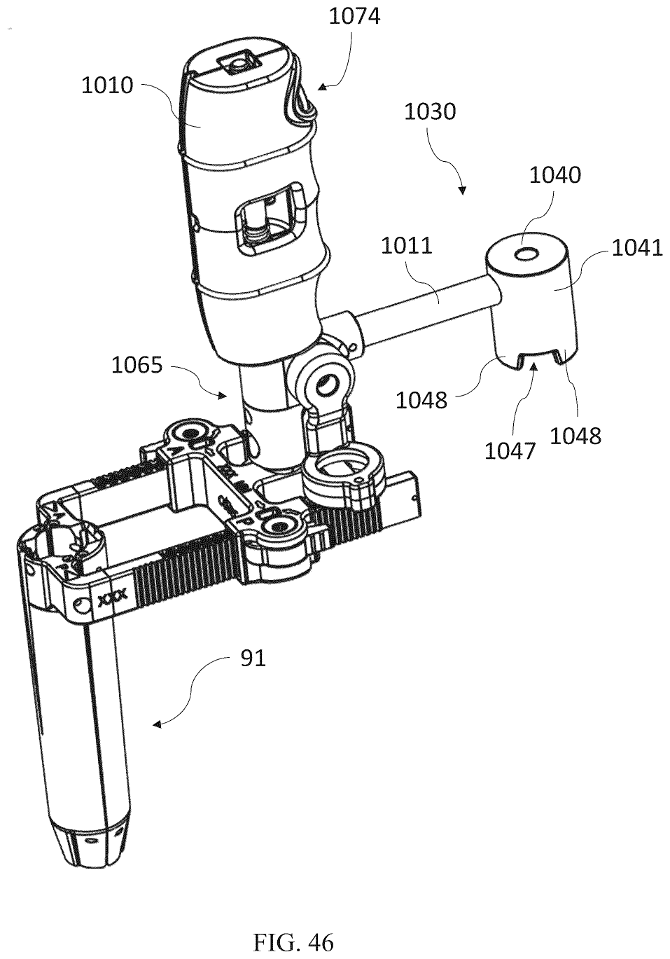

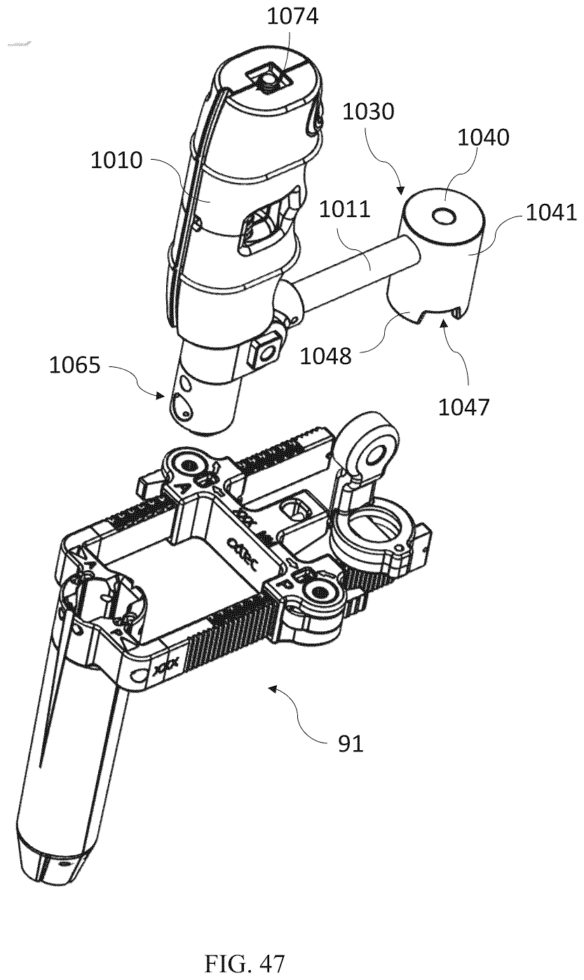

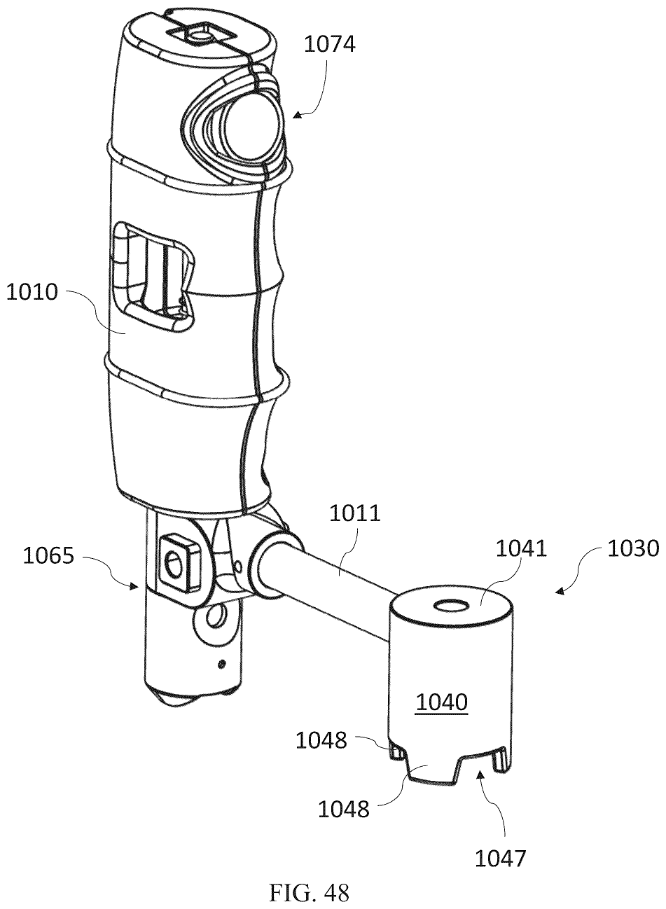

FIGS. 46 through 48 illustrate another embodiment of a handle 1010 and drape connector 1030 for connecting a surgical instrument 91 to the mechanical arm 1000 of FIGS. 1 and 2, with a surgical instrument 91 attached to the handle 1010. In this embodiment, the handle 1010 connects directly to the housing 1040 of the drape connector 1030 through a link 1011. Similar to the drape connector 1030 includes a flexible collet (not shown) that allows the housing 1040 to engage and connect to the connection shank 131′ illustrated in FIGS. 44A and 44B.

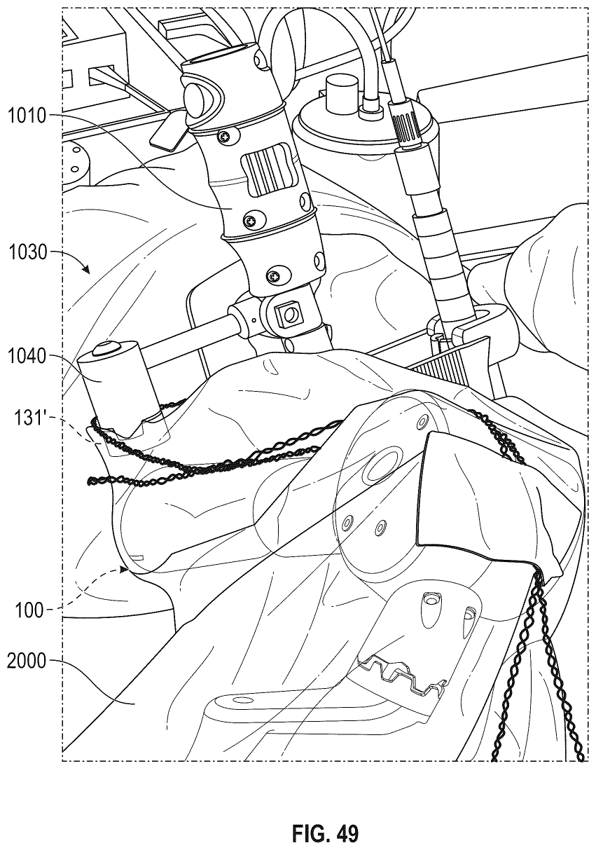

Specifically, similar to the housings 140, 140′, the housing 1040 has a body 1041 including a plurality of teeth or projections 1048 defining a plurality of cut-outs 1047 positioned at a distal end of the housing 1040. The plurality of projections 1048 and plurality of cut-outs 1047 mechanically engage and interface with the plurality of teeth 136′ and the plurality of gaps 137′ of the connection shank 131′ (see FIGS. 44A and 44B). For example, FIG. 49 illustrates the handle 1010 and drape connector 1030 connected to the distal joint 100 across a sterile drape 2000 through the connection shank 131′.

Although not illustrated, the drape connector 1030 includes a flexible collect within the housing 1040 the extends toward the distal end of the housing 1040 with a proximal portion of the flexible collect extending proximally through the hole shown at the proximal end of the housing 1040. The proximal portion of the flexible collet may be translated proximally by a thumbwheel or lock, such as the lock 160′ or the lock 160, or a similar screw-type configuration capable of withdrawing proximally the proximal portion of the flexible collet so to lock the distal end of the collet onto the connection shank 131′.

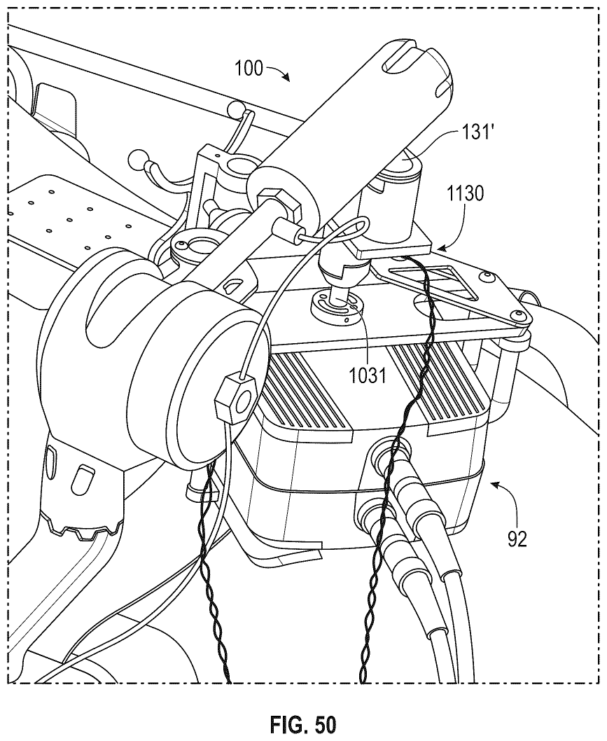

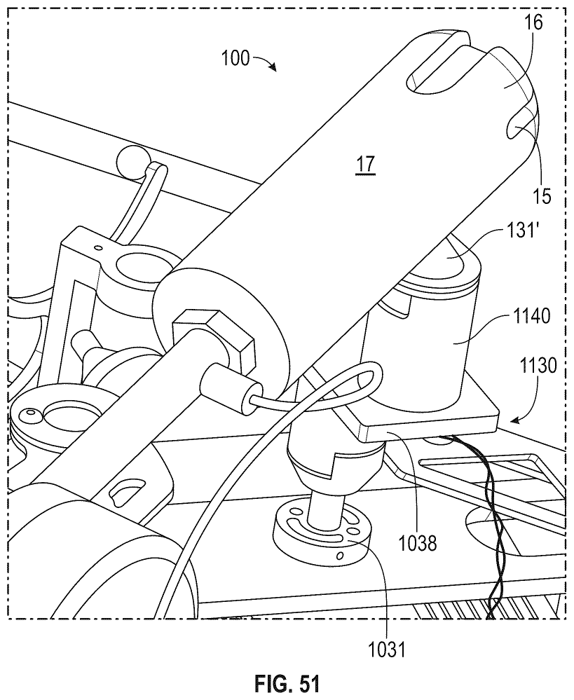

FIGS. 50 and 51 illustrate another embodiment of a drape connector 1130 for connecting a surgical instrument 92 to the mechanical arm 1000 of FIGS. 1 and 2, with the surgical instrument 92 attached to the drape connector 1130. The drape connector 1130 includes the connection shank 131′ of FIGS. 44A and 44B, a housing 1140 connectable to the connection shank 131′, an intermediate connector 1038, and a second connection shank 1031.

The housing 1140 may be the housing 1040 of FIGS. 46 through 48. Alternatively, the housing 1140 may be similar to the housing 1040 of FIGS. 46 through 48, and may mechanically engage the connection shank 131′ through a plurality of projections and a plurality of gaps. As before, the connection shank 131′ may be engaged by the inner clamp 15 of the distal joint 100. Also as before, introduction of high-pressure fluid (e.g., CO₂) into the distal joint 100 (e.g., the pneumatic assembly 18) allows the outer clamp 16 to move relative to the housing 17, thereby allowing movement of the surgical instrument 92.

The housing 1140 is connectable to the intermediate connector 1038 at an end opposite from where the housing 1140 connects to the connection shank 131′. The housing 1140 may connect to the intermediate connector 1038 such that the housing 1140 may rotate relative to the intermediate connector 1038. The intermediate connector 1038 may also be connected to the second connection shank 1031 by way of a second housing similar, in some embodiments, to the housing 1140, although not visible in FIG. 51. Thus, the drape connector 1130 may have two sets of housings, two sets of flexible collects, and two sets of teeth/gaps for non-rotational engagement. In some embodiments, the drape connector 1130 also has two sets of locks or thumbwheels configured to cause each flexible collet, independently, to lock onto a respective connection shank. In some embodiments, the drape connector 1130 has only a single lock or thumbwheel that is configured to simultaneously cause the flexible collects to lock onto respective connection shanks.

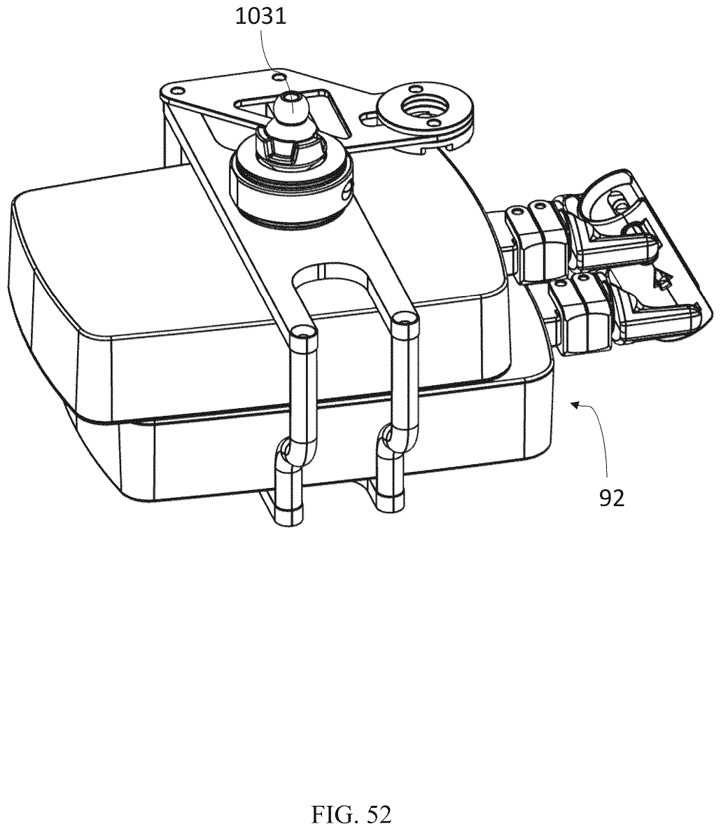

Referring briefly to FIG. 52, the second connection shank 1031 may be incorporated into the surgical instrument 92.

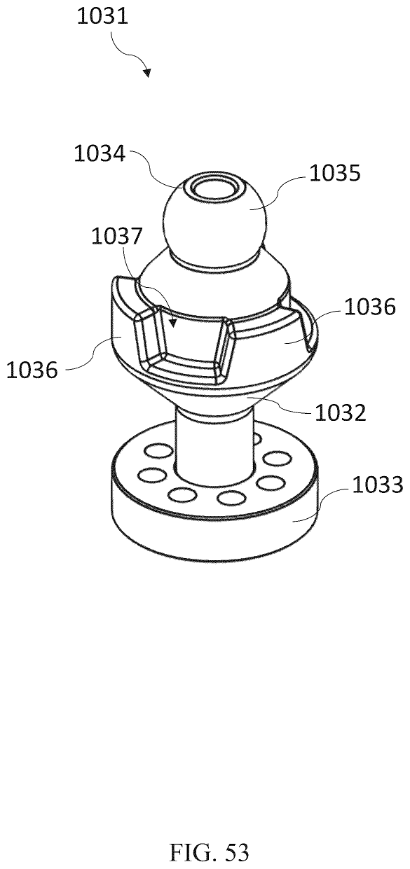

Alternatively, the second connection shank 1031 may be a separate component that is connectable to the surgical instrument 92. For example, FIG. 53 illustrates the second connection shank 1031 for connecting the surgical instrument 92 with the distal joint 100 of the mechanical arm 1000 of FIGS. 1 and 2. Similar to the connection shanks 131, 131′, the second connection shank 1031 includes a body 1032 having a first end 1033 in connection with the surgical instrument 92 and a second end 1034 in connection with a ball join 1035. The body 1032 also includes a plurality of teeth 1036 defining a plurality of gaps 1037. The plurality of teeth 1036 and the plurality of gaps 1037 mechanically engage with the intermediate connector 1038, thereby securing the second connection shank 1031 and the surgical instrument 92 to the distal joint 100 and the mechanical arm 1000.

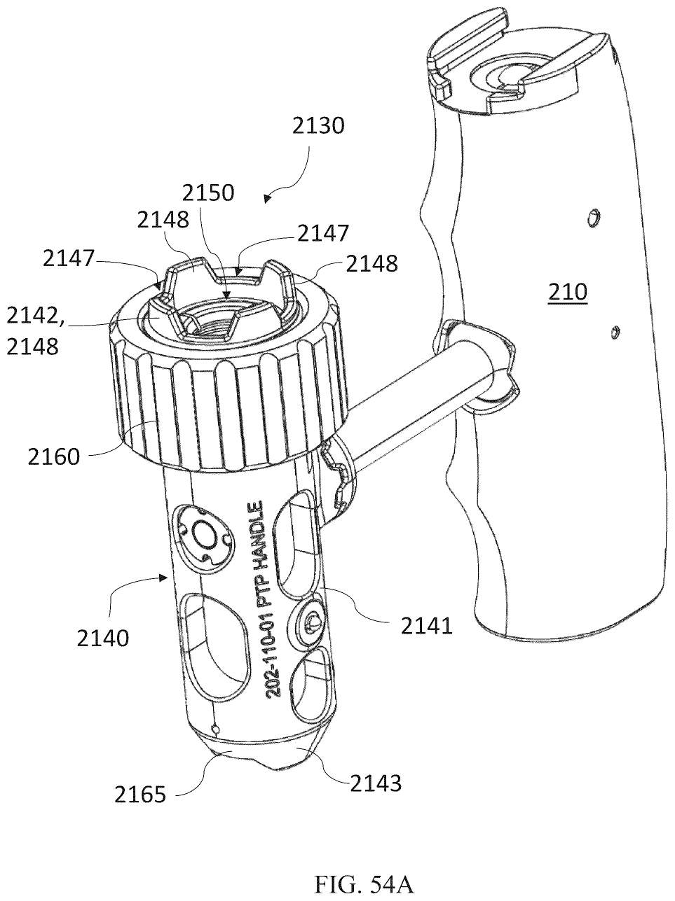

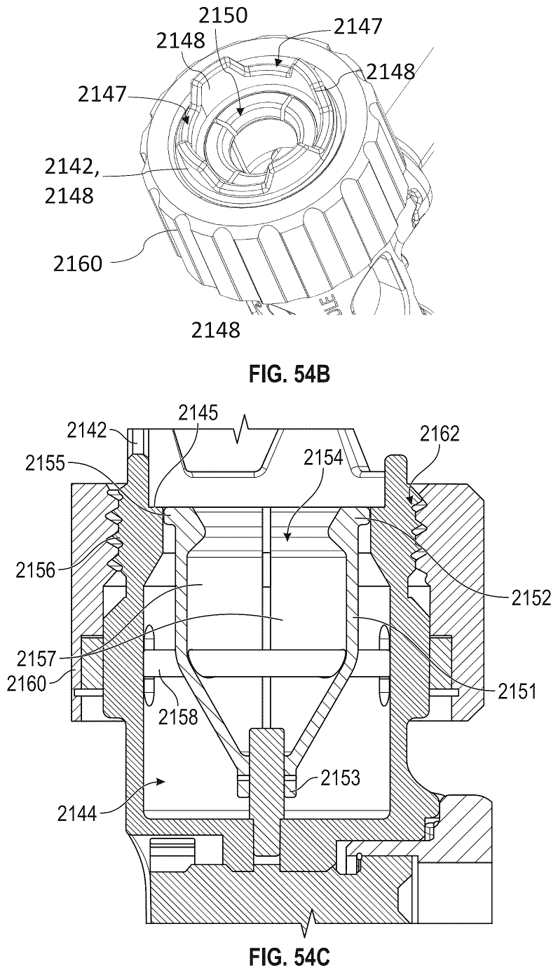

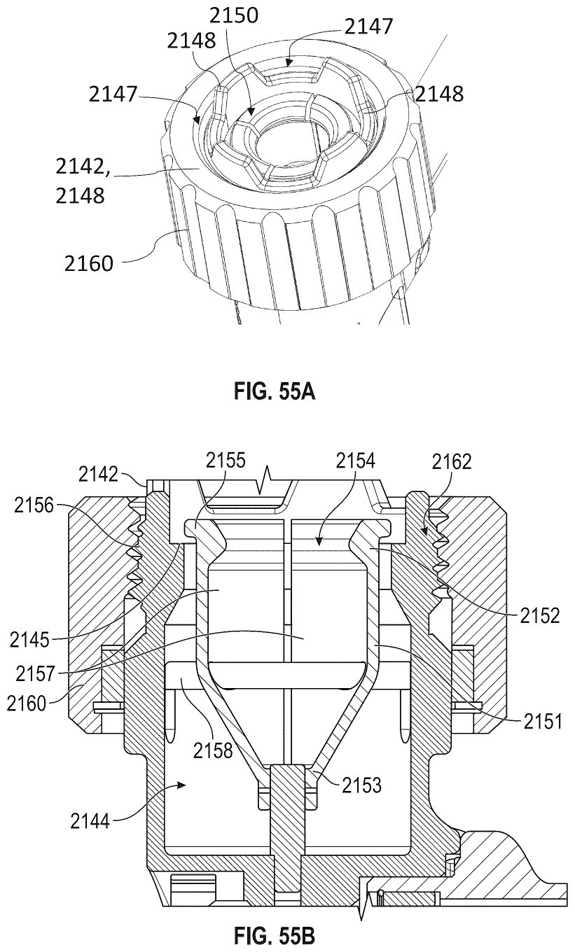

FIGS. 54A through 55B illustrate another embodiment of a handle 210 and drape connector 2130 for connecting a surgical instrument to a mechanical arm, such as the mechanical arm of FIGS. 1 and 2. The drape connector 2130 is for attaching a surgical tool to the distal joint 100 across a sterile drape. The drape connector 2130 attaches to the tool connection shank 131, 1031 in association with the mechanical arm 1000. Specifically, the connection shank 131 is associated with or in connection with the inferior end 13 of the link 11 (see FIG. 4). The drape connector 2130 attaches to the connection shank 131 across the sterile drape, such that the drape connector 2130 is outside the sterile drape.

The drape connector 2130 includes a housing 2140, a flexible collet 2150, and an instrument mounting interface 2165. The housing 2140 includes a body 2141 extending between a superior end 2142 and an inferior end 2143. The superior end 2142 may be crenellated, with a plurality of teeth or projections 2148 that define a plurality of cut-outs 2147. When the connection shank 131 engages the housing 2140 (for example, see FIGS. 13 to 16), the plurality of teeth 136 are received within the plurality of cut-outs 2147 and the plurality of projections 2148 are received within the plurality of gaps 137. In this way, the connection shank 131 is mechanically engaged with the housing 2140. The engagement of the cut-outs and projections creates multiple contact surfaces which limit movement of the connection shank 131 (and therefore movement of the mechanical arm 1000) relative to the housing 2140 of the drape connector 2130.

Similar to the drape connector 2130, when the connection shank 131 is engaged with the housing 2140, the ball 135 of the connection shank 131 is received and engaged by a flexible collet 2150, which is positioned within the housing 2140, across the sterile drape. Referring to FIGS. 54C and 55B, the flexible collet 2150 has a body 2151 extending between a superior end 2152 and an inferior end 2153. Extending towards the superior end 2152 are a flange 2155, and a plurality of prongs 2157 that form a receptacle 2154 and. The plurality of prongs 2157 flex outwardly as the ball 135 is positioned within the receptacle 2154 and then snap back around the ball 135 to seat the ball 135 within the receptacle 2154.

The flexible collet 2150 is adjustable between an unlocked position and a locked position through action of a lock 2160 associated with the superior end 2152 of the housing 2140. Referring to FIG. 54C, the flexible collet 2150 is positioned within a channel 2144 defined by the body 2141 of the housing 2140. Disposed near the superior end 2142 of the housing 2140 is threading 2162, that engages threading of the lock 2160, and an internal ledge 2145. The receptacle 2154 and the flange 2155, formed by the plurality of prongs 2157 of the flexible collet 2150, are positioned near the superior end 2142 of the housing 2140 when the flexible collet 2150 is received within the housing 2140 in an unlocked position (see FIG. 55B). Once the ball 135 is seated within the receptacle 2154, the lock 2160 may be actuated to draw the flexible collet 2150 inferiorly within the housing 2140.

For example, actuation of the lock 2160 pulls the flexible collet 2150 inwardly within the housing 2140. As the flexible collet 2150 is pulled inwardly (FIG. 54C), the plurality of prongs 2157 move from an extended configuration to a retracted configuration or locked position. Additionally, the flange 2155 is pulled past the internal ledge 2145 of the housing 2140. This tightens the receptacle 2154 around the ball 135 and prevents separation of the connection shank 131 (e.g., the ball 135) and the housing 2140 (e.g., the flexible collet 2150). The flexible collet 2150 is anchored within the housing 2140 through a dowel pin 2158.

The lock 2160 is associated with a lock interface 2156. As illustrated, the lock interface 2156 includes threads 2162 and the lock 2160 is a wheel moveable along the threads 2162. The lock 2160 may be placed at any position desired, and in some embodiments, the lock 2160 is externally accessible near the superior end 2142 of the housing 2140. The lock 2160 may be quickly and easily actuated by a practitioner using one hand. In other configurations, the lock 2160 may be in connection with the lock interface 2156 and located at a different position on the housing 2140 (e.g., in the middle, near the inferior end 2143, etc.). For example, the lock 2160 may be configured to be actuated by a surgeon's thumb, or rotated by a surgeon's hand, etc.

A surgical tool or instrument may be connected to the housing 2140 through the instrument mounting interface 2165. Similar to the instrument mounting interface 165, the instrument mounting interface 2165 may include a rod for receiving the surgical tool or instrument and a spring. The spring biases the rod in a locked configuration. Attachment of the surgical tool to the rod compresses the spring, allowing the surgical tool to be connected to the rod. Upon connection, the spring will rebound and secure the surgical tool to the inferior end 2143 of the housing 2140. The surgical tool may now be maneuvered through motions of the distal joint 100 (e.g., rotation, angular adjustment, and forward and backward translation).

### EMBODIMENTS

Embodiment 1. A drape connector for securing a tool to a surgical arm across a sterile drape, the drape connector comprising a flexible collet for receiving a ball of a tool connection shank of the surgical arm to selectively secure the flexible collet to the surgical arm; a lock associated with an inferior end of the flexible collet, the lock for adjusting the flexible collet from an unlocked position to a locked position; and a housing for receiving the flexible collet and the lock.
Embodiment 2. The drape connector of Embodiment 1, further comprising an instrument mounting interface at an inferior end of the housing, the instrument mounting interface for connecting a surgical instrument to the drape connector.
Embodiment 3. The drape connector of Embodiment 2, wherein the instrument mounting interface comprises a spring-loaded rod.
Embodiment 4. The drape connector of any one of Embodiments 1, 2, or 3, wherein the flexible collet comprises a body extending toward a superior end; a receptacle defined by the body and forming the flexible collet, the receptacle open at the superior end for receiving the ball of the tool connection shank; a flange at the superior end, the flange for engaging an internal ledge of the housing in the locked position; and a lock interface extending between the receptacle and the inferior end, the lock interface for receiving and engaging the lock.
Embodiment 5. The drape connector of Embodiment 4, wherein the body comprises a plurality of teeth/prongs forming the receptacle and the flange, the plurality of teeth/prongs flexible about the internal ledge of the housing as the flexible collet is adjusted from the unlocked position to the locked position.
Embodiment 6. The drape connector of either one of Embodiments 4 or 5, wherein the lock interface comprises threading and the lock comprises a wheel actuatable along the threading.
Embodiment 7. The drape connector of any one of Embodiments 1, 2, 3, 4, 5, or 6, wherein the flexible collet is in an extended configuration when the flexible collet is the unlocked position and the flexible collet is pulled inwardly within the housing to a retracted configuration when the flexible collet is in the locked position.
Embodiment 8. The drape connector of any one of Embodiments 1, 2, 3, 4, 5, 6, or 7, wherein the housing comprises a body extending between a superior end and an inferior end; a channel defined by the body and extending between the superior end and the inferior end; and an internal ledge disposed near the superior end, the internal ledge for interfacing with the superior end of the flexible collet in the unlocked position and the locked position.
Embodiment 9. The drape connector of Embodiment 8, wherein the superior end of the body defines a plurality of cut-outs corresponding to and for receiving a plurality of teeth of the tool connection shank.
Embodiment 10. The drape connector of Embodiment 9, wherein the plurality of cut-outs are irregularly spaced around a circumference of the superior end of the body.
Embodiment 11. The drape connector of either one of Embodiments 8 or 9, wherein each cut-out in the plurality of cut-outs is sized differently from the other cut-outs
Embodiment 12. The drape connector of any one of Embodiments 8, 9, 10, or 11, wherein the lock comprises a wheel, the wheel being accessible through and actuatable within a window.
Embodiment 13. The drape connector of any one of Embodiments 1, 2, 3, 4, 5, 6, 7, 8, 9, 10, 11, or 12, wherein the ball is secured to a superior end of the flexible collet with a snap-fit engagement.
Embodiment 14. The drape connector of any one of Embodiments 1, 2, 3, 4, 5, 6, 7, 8, 9, 10, 11, 12, or 13, wherein the engagement between the collet and the ball of the tool connection shank does not puncture or tear the sterile drape.
Embodiment 15. The drape connector of any one of Embodiments 1, 2, 3, 4, 5, 6, 7, 8, 9, 10, 11, 12, 13, or 14, wherein the housing comprises a window for providing external access to the lock, the window defined by the body and extending from an exterior of the body.
Embodiment 16. The drape connector of any one of Embodiments 1, 2, 3, 4, 5, 6, 7, 8, 9, 10, 11, 12, 13, or 14, wherein the lock is positioned on an exterior surface of the housing.
Embodiment 17. A method of connecting a surgical tool to a surgical arm, the method comprising draping the surgical arm with a sterile drape; attaching a first end of a drape connector to the surgical arm and over the sterile drape, the drape connector having a flexible collet in an open position; actuating a lock of the drape connector, such that the flexible collet is in a closed position and locked to the surgical arm; and attaching the surgical tool to a second end of the drape connector opposite from the first end.
Embodiment 18. The method of Embodiment 17, wherein attaching the drape connector to the surgical arm and locking the drape connector does not tear or puncture the sterile drape.
Embodiment 19. A system for connecting a drape to a surgical arm, the system comprising: a surgical arm comprising a tool connection shank comprising a ball at an end of the surgical arm; a drape for placing between the tool connection shank and a drape connector; the drape connector for securing a tool the surgical arm, the drape connector comprising a flexible collet for selectively receiving the ball of the tool connection shank to secure the drape connector to the surgical arm across the drape; a lock for adjusting the flexible collect from an unlocked position to a locked position; and a housing for receiving the flexible collet and the lock.
Embodiment 20. The system of claim 19, wherein the tool connection shank comprises: a body having one end in connection with the surgical arm and the other end in connection with the ball; and a plurality of teeth disposed about the body, the plurality of teeth defining a corresponding plurality of gaps, the plurality of teeth for mechanically engaging the housing, wherein the ball inferiorly extends from the body and past the plurality of teeth for engaging with the flexible collet.
Embodiment 21. A mechanical arm for use in surgery, the mechanical arm comprising a distal joint comprising a handle and a drape connector, the drape connector for connecting a surgical instrument to the mechanical arm across a sterile drape, the distal joint having three (3) degrees of freedom allowing the surgical instrument to be rotated, translated forward and backward, and angularly adjusted; a medial joint connected to the distal joint, the medial joint having one (1) degree of freedom allowing the distal joint to pivot about the medial joint; a translating link connected to the medial joint and extending inferiorly away from the distal joint, the translating link having one (1) degree of freedom allowing a length of the mechanical arm to be adjusted; and a proximal joint connected to the translating link, the proximal joint having two (2) degrees of freedom, the proximal joint for connecting the mechanical arm to a surgical table, each of the distal joint, the medial joint, and the proximal joint transitional between a default locked state and an open state.
Embodiment 22. The mechanical arm of Embodiment 21, wherein the distal joint comprises the handle connected to the drape connector through a link, the handle configured to receive a high pressure fluid line; a ball disposed on the link between the handle and the drape connector; a clamp for receiving the ball, the ball rotatable and angularly adjustable within the clamp; a pneumatic assembly connected between the clamp and the medial joint, the pneumatic assembly having a pneumatic actuator and at least one spring; and a housing for receiving the pneumatic assembly and a portion of the clamp, the portion of the clamp rotatable relative to the housing.
Embodiment 23. The mechanical arm of Embodiment 22, wherein the pneumatic actuator of the pneumatic assembly comprises a piston, the piston actuated when the high pressure fluid line is opened.
Embodiment 24. The mechanical arm of either one of Embodiments 22 or 23, wherein the distal joint further comprises a button disposed at a superior end of the handle, the button for selectively opening the high pressure fluid line.
Embodiment 25. The mechanical arm of any one of Embodiments 22, 23, or 24, wherein the high pressure fluid line comprises a high pressure CO₂ line.
Embodiment 26. The mechanical arm of any one of Embodiments 22, 23, 24, or 25, wherein the at least one spring comprises a compressible Belleville disc.
Embodiment 27. The mechanical arm of any one of Embodiments 22, 23, 24, 25, or 26, wherein compression of the at least one spring upon actuation of the pneumatic actuator allows the portion of the clamp to rotate relative to the housing.
Embodiment 28. The mechanical arm of any one of Embodiments 21, 22, 23, 24, 25, 26, or 27, wherein the medial joint comprises a medial joint yoke; a floating taper flange received by the medial joint yoke and connectable to the distal joint; a fixed taper flange in communication with the floating taper flange; and an actuator in connection with the medial joint yoke, the actuator in mechanical communication with the fixed taper flange and the floating taper flange, wherein when the medial join is in the open state, the floating taper is separated from the fixed taper allowing the distal joint to pivot about the medial joint.
Embodiment 29. The mechanical arm of claim 28, wherein the actuator comprises a piston in connection with the medial joint yoke.
Embodiment 30. The mechanical arm of either one of Embodiments 28 or 29, further comprising at least one compression spring and a spring compressor received by the floating taper flange, the spring compressor actuatable by the actuator.
Embodiment 31. The mechanical arm of any one of Embodiments 28, 29, or 30, wherein the floating taper flange is rotatable relative to the fixed taper flange with the medial joint is in the open state.
Embodiment 32. A method of using a mechanical surgical arm, the method comprising opening a high pressure fluid line to actuate an actuator within a distal joint, causing a housing of the distal joint to mechanically separate from a clamp of the distal joint and allowing three (3) degrees of freedom for the distal joint; compressing a spring of a medial joint, the medial joint connected to the distal joint, compression of the spring causing a fixed taper flange of the medial joint to mechanically separate from a floating taper flange of the medial joint and allowing the distal joint to pivot about the medial joint; and rotating a surgical instrument connected to the distal joint.
Embodiment 33. The method of Embodiment 32, further comprising sterilely attaching the surgical instrument to the distal joint, the surgical instrument attached to the distal joint across a sterile drape. The embodiments of any of the embodiments 1-32 may allow the mechanical arm to be adjusted with one hand.
Embodiment 34. The method of either one of Embodiments 32 or 33, wherein actuation of the actuator comprises delivering a volume of fluid through the high-pressure fluid line to a piston; pneumatically actuating the piston, such that the piston pushes on a spring compressor; and compressing a spring, such that the housing of the distal joint separates from the clamp of the distal joint, allowing the clamp to rotate relative to the housing.
Embodiment 35. The method of any one of Embodiments 32, 33, or 34, wherein a handle of the distal joint is connected to the surgical instrument through a link, and rotating a surgical instrument connected to the distal joint comprises moving the handle, such that a spherical component disposed on the link between the handle and the surgical instrument moves within the clamp.
Embodiment 36. The method of any one of Embodiments 32, 33, 34, or 35, wherein pivoting the distal joint about the medial joint can be accomplished with one hand.
Embodiment 37. The method of any one of Embodiments 32, 33, 34, 35, or 36, wherein opening a high-pressure fluid line comprises actuating an opener disposed on a handle of the distal joint and flowing high pressure fluid into the high pressure fluid line.
Embodiment 38. A method of attaching a surgical instrument to a mechanical arm, the method comprising aligning a receiver of the mechanical arm over a basket, the basket held by a housing; joining the receiver to the basket, the receiver contained within a sterile drape and the basket external to the sterile drape, the receiver joined to the basket across the sterile drape; locking the basket to the receiver; and attaching a surgical arm to the housing.
Embodiment 39. The method of Embodiment 38, wherein aligning a receiver of the mechanical arm over a basket comprises aligning a plurality of teeth of the receiver relative to a crenellated superior end of the housing, such that the plurality of teeth will be received by crenels of the superior end.
Embodiment 40. The method of either one of Embodiments 38 or 39, further comprising forming a partial kinematic lock between the receiver and the basket.
Embodiment 41. The method of any one of Embodiments 38, 39, or 40, wherein joining the receiver to the basket comprises pressing an extension of the receiver into a collection receptacle of the basket, the collection receptacle formed from a plurality of prongs; flexing the plurality of prongs about the extension to receive the extension within the collection receptacle; and snapping the plurality of prongs about the extension.
Embodiment 42. The method of any one of Embodiments 38, 39, 40, or 41, wherein locking the basket to the receiver comprises actuating a lock in connection with the basket; pulling the basket into the housing; and snapping a superior edge of the basket over an internal ledge of the housing, such that the basket is locked within the housing.
Embodiment 43. The method of any one of Embodiments 38, 39, 40, 41, or 42, wherein attaching a surgical arm to the housing comprises inserting an attachment end of the surgical instrument into an instrument mounting interface of the housing; compressing a spring of the instrument mounting interface; and expanding the spring, thereby securing the surgical instrument to the instrument mounting interface, the surgical instrument being rotatable, angularly moveable, and moveable in a forward and backward direction.
Embodiment 44. A mechanical arm for use in surgery, the mechanical arm comprising a distal joint comprising a handle and a drape connector, the drape connector for connecting a surgical instrument to the mechanical arm across a sterile drape, the distal joint having three (3) degrees of freedom allowing the surgical instrument to be rotated, translated forward and backward, and angularly adjusted; a medial joint connected to the distal joint, the medial joint having one (1) degree of freedom allowing the distal joint to pivot about the medial joint; a translating link connected to the medial joint and extending inferiorly away from the distal joint, the translating link having one (1) degree of freedom allowing a length of the mechanical arm to be adjusted; and a proximal joint connected to the translating link, the proximal joint having two (2) degrees of freedom, the proximal joint for connecting the mechanical arm to a surgical table, each of the distal joint, the medial joint, and the proximal joint comprising a fixed and a floating taper locked together by spring forces, and transitional between a default locked state and an open state only by pneumatic actuators using high pressure CO₂ to remove compression on the floating taper and move each of the distal joint, the medial joint, and the proximal joint to the open state.
Embodiment 45. A drape connector for sterilely connecting a tool to a surgical arm having a tool connection shank, the drape connector comprising a flexible collet for receiving a ball of the tool connection shank to selectively secure the flexible collet to the surgical arm across a sterile drape; a lock associated with an inferior end of the flexible collet, the lock for adjusting the flexible collet from an unlocked position to a locked position; and a housing for receiving the flexible collet and the lock, a superior end of the housing having a plurality of teeth and a plurality of cut-outs, the superior end of the housing for mechanically engaging the tool connection shank, such that the superior end of the housing and the tool connection shank have six points of contact between the superior end of the housing and the tool connection shank.
Embodiment 46. A drape connector for sterilely connecting a tool to a surgical arm, the drape connector comprising: (a) a first drape connector as in any one of Embodiments 1, 2, 3, 4, 5, 6, 7, 8, 9, 10, 11, 12, 13, 14, 15, or 16, the first drape connector orientated in a first direction defined by the flexible collet of the first drape connector; (b) a second drape connector as in any one of Embodiments 1, 2, 3, 4, 5, 6, 7, 8, 9, 10, 11, 12, 13, 14, 15, or 16, the second drape connector orientated in a second direction defined by the flexible collet of the second drape connector; wherein the first and second directions are not the same.
Embodiment 47. The drape connector of Embodiment 46, wherein the first and second directions are parallel but not coaxial.
Embodiment 48. The drape connector of Embodiment 46, wherein the first and second directions are opposite but not coaxial.
Embodiment 49. The drape connector of Embodiment 46, wherein the first and second directions are opposite and coaxial.
Embodiment 50. The drape connector of any one of Embodiments 46, 47, 48, or 49, wherein the respective housings of the first and second drape connectors are integrally formed so as to constitute a single housing.
Embodiment 51. The drape connector of any one of Embodiments 46, 47, 48, 49, or 50, wherein the respective locks operate independently of each other to adjust the respective flexible collets.
Embodiment 52. The drape connector of any one of Embodiments 46, 47, 48, 49, 50, or 51, wherein the respective locks operate in unison with each other to simultaneously adjust the respective flexible collets.

### Additional Terms and Definitions

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It should also be noted that some of the embodiments disclosed herein may have been disclosed in relation to a particular surgical applications (e.g., spinal procedures); however, other applications (e.g., spinal procedures for various approaches such as PTP, ALIF, LTP, etc., as well as other, non-spinal surgical applications, etc.) are also contemplated. In other configurations, one, two, three, or more joints may be actuated joints. Components may be modified in length, width, etc., to account for different surgical needs. For example, the arm may be given a different range of motion, different surgeon touch points, and different load capacities depending on the surgical needs.

In one embodiment, the terms “about” and “approximately” refer to numerical parameters within 10% of the indicated range. The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the author(s) of this disclosure for carrying out the embodiments disclosed herein. Of course, variations on these described embodiments will become apparent to those of ordinary skill in the art upon reading the foregoing description. The author(s) expects skilled artisans to employ such variations as appropriate, and the author(s) intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A drape connector for securing a tool to a surgical arm across a sterile drape, the drape connector comprising:
a flexible collet for receiving a ball of a connection shank of the surgical arm to selectively secure the flexible collet to the surgical arm;
a lock associated with an inferior end of the flexible collet, the lock for adjusting the flexible collet from an unlocked position to a locked position; and
a housing for receiving the flexible collet and the lock.

2. The drape connector of claim 1, further comprising an instrument mounting interface at an inferior end of the housing, the instrument mounting interface for connecting a surgical instrument to the drape connector.

3. The drape connector of claim 2, wherein the instrument mounting interface comprises a spring-loaded rod.

4. The drape connector of claim 1, wherein the flexible collet comprises:
a body extending toward a superior end;
a receptacle defined by the body and forming the flexible collet, the receptacle open at the superior end for receiving the ball of the connection shank;
a flange at the superior end, the flange for engaging an internal ledge of the housing in the locked position; and
a lock interface extending between the receptacle and the inferior end, the lock interface for receiving and engaging the lock.

5. The drape connector of claim 4, wherein the body comprises a plurality of prongs forming the receptacle and the flange, the plurality of prongs flexible about the internal ledge of the housing as the flexible collet is adjusted from the unlocked position to the locked position.

6. The drape connector of claim 4, wherein the lock interface comprises threading and the lock comprises a wheel actuatable along the threading.

7. The drape connector of claim 1, wherein the flexible collet is in an extended configuration when the flexible collet is the unlocked position and the flexible collet is pulled inwardly within the housing to a retracted configuration when the flexible collet is in the locked position.

8. The drape connector of claim 1, wherein the housing comprises:
a body extending between a superior end and an inferior end;
a channel defined by the body and extending between the superior end and the inferior end; and
an internal ledge disposed near the superior end, the internal ledge for interfacing with the superior end of the flexible collet in the unlocked position and the locked position;
a window for providing external access to the lock, the window defined by the body and extending from an exterior of the body to the channel.

9. The drape connector of claim 8, wherein the superior end of the body defines a plurality of cut-outs corresponding to and for receiving a plurality of teeth of the connection shank.

10. The drape connector of claim 9, wherein the plurality of cut-outs are irregularly spaced around a circumference of the superior end of the body.

11. The drape connector of claim 8, wherein each cut-out in the plurality of cut-outs is sized differently from the other cut-outs.

12. The drape connector of claim 8, wherein the lock comprises a wheel, the wheel being accessible through and actuatable within the window.

13. The drape connector of claim 1, wherein the ball is secured to a superior end of the flexible collet with a snap-fit engagement.

14. The drape connector of claim 1, wherein the engagement between the collet and the ball of the connection shank does not puncture or tear the sterile drape.

15. A system for connecting a drape to a surgical arm, the system comprising:
a surgical arm comprising a connection shank comprising a ball at an end of the surgical arm;
a drape for placing between the connection shank and a drape connector;
the drape connector for securing a tool the surgical arm, the drape connector comprising:
a flexible collet for selectively receiving the ball of the connection shank to secure the drape connector to the surgical arm across the drape;
a lock for adjusting the flexible collect from an unlocked position to a locked position; and
a housing for receiving the flexible collet and the lock.

16. The system of claim 15, wherein the connection shank comprises:
a body having one end in connection with the surgical arm and the other end in connection with the ball; and
a plurality of teeth disposed about the body, the plurality of teeth defining a corresponding plurality of gaps, the plurality of teeth for mechanically engaging the housing,
wherein the ball inferiorly extends from the body and past the plurality of teeth for engaging with the flexible collet.

17. A drape connector for sterilely connecting a tool to a surgical arm having a shank, the drape connector comprising:
a flexible collet for receiving a ball of the connection shank to selectively secure the flexible collet to the surgical arm across a sterile drape;
a lock associated with an inferior end of the flexible collet, the lock for adjusting the flexible collet from an unlocked position to a locked position; and
a housing for receiving the flexible collet and the lock, a superior end of the housing having a plurality of teeth and a plurality of cut-outs,
the superior end of the housing for mechanically engaging the connection shank, such that the superior end of the housing and the connection shank have six points of contact between the superior end of the housing and the connection shank.

18. The drape connector of claim 17, further comprising an instrument mounting interface at an inferior end of the housing, the instrument mounting interface for connecting a surgical instrument to the drape connector.

19. The drape connector of claim 17, wherein the flexible collet is in an extended configuration when the flexible collet is the unlocked position and the flexible collet is pulled inwardly within the housing to a retracted configuration when the flexible collet is in the locked position.

20. The drape connector of claim 17, wherein the lock is accessible from an exterior of the housing.
